SEQUENCE LISTING (12 sequences)

SEQ ID NO: 1 - DNA, 27 bp, Homo sapiens
  source 1..27: mol_type = other DNA
tgtactgcagagataagtttagctgac

SEQ ID NO: 2 - DNA, 23 bp, Homo sapiens
  source 1..23: mol_type = other DNA
tccttgtaagtggcttcttgaac

SEQ ID NO: 3 - DNA, 20 bp, Homo sapiens
  source 1..20: mol_type = other DNA
gtgaggttgctcatcggttt

SEQ ID NO: 4 - DNA, 20 bp, Homo sapiens
  source 1..20: mol_type = other DNA
gagcaaaggcaatcaacacc

SEQ ID NO: 5 - DNA, 20 bp, Homo sapiens
  source 1..20: mol_type = other DNA
ggcacagctggaggtcttat

SEQ ID NO: 6 - DNA, 21 bp, Homo sapiens
  source 1..21: mol_type = other DNA
tgttcaggcatcagagaagtg

SEQ ID NO: 7 - DNA, 21 bp, Homo sapiens
  source 1..21: mol_type = other DNA
tggatcctgagaacttcaagc

SEQ ID NO: 8 - DNA, 19 bp, Homo sapiens
  source 1..19: mol_type = other DNA
gccactgcagtcaccatct

SEQ ID NO: 9 - DNA, 18 bp, Homo sapiens
  source 1..18: mol_type = other DNA
gaagcacctccggaacct

SEQ ID NO: 10 - DNA, 20 bp, Homo sapiens
  source 1..20: mol_type = other DNA
gtcacctcgttcatgcactc

SEQ ID NO: 11 - DNA, 21 bp, Homo sapiens
  source 1..21: mol_type = other DNA
cccggggttctatgatatttg

SEQ ID NO: 12 - DNA, 22 bp, Homo sapiens
  source 1..22: mol_type = other DNA
gccctgaagaaagtcctctaca

(19) United States
(12) Patent Application Publication
(10) Pub. No.: US 2026/0117189 A1
(43) Pub. Date: Apr. 30, 2026

(54) METHODS OF GENERATING AND EXPANDING HEMATOPOIETIC STEM CELLS

(71) Applicant: CHILDREN'S HOSPITAL MEDICAL CENTER, Cincinnati, OH (US)

(72) Inventors: Takanori TAKEBE, Cincinatti, OH (US); Kyle LEWIS, Cincinatti, OH (US)

(73) Assignee: CHILDREN'S HOSPITAL MEDICAL CENTER, Cincinnati, OH (US)

(21) Appl. No.: 19/387,157

(22) Filed: Nov. 12, 2025

Related U.S. Application Data
(63) Continuation of application No. 17/595,493, now Pat. No. 12,497,597.
(60) Provisional application No. 62/855,536, filed on May 31, 2019.
(60) Provisional application No. 62/855,524, filed on May 31, 2019.

(51) Int. Cl. C12N 5/071; A61K 35/12; A61K 35/28; C12N 5/0789

(52) U.S. Cl. CPC C12N 5/0671; A61K 35/12; A61K 35/28; C12N 5/0647; C12N 2501/119; C12N 2501/155; C12N 2501/16; C12N 2501/727; C12N 2506/45; C12N 2533/90

(57) ABSTRACT

Production and maintenance of hematopoietic stem cells in in vitro culture systems has proven to be elusive. Disclosed herein are hematopoietic stem cell compositions that originate from organoids such as liver organoids. These organoids also comprise a rare type of immune cell that is not yet fully elucidated due to the difficulty in isolating said immune cell from biological samples. Also disclosed herein are methods of producing said hematopoietic stem cells and immune cells from organoids, as well as methods of expanding hematopoietic stem cells from other sources using these organoids.

### CROSS-REFERENCE TO RELATED APPLICATIONS

This application is a Continuation application from U.S. Non-Provisional Ser. No. 17/595,493, filed Nov. 17, 2021, which is a National Stage Entry of PCT/US2020/035385, filed May 29, 2020, which claims the benefit of priority of U.S. Provisional Patent Application No. 62/855,524, filed May 31, 2019 and U.S. Provisional Patent Application No. 62/855,536, filed May 31, 2019, each of which is hereby expressly incorporated by reference in its entirety.

### REFERENCE TO SEQUENCE LISTING

The present application is being filed along with a Sequence Listing in electronic format. The Sequence Listing is provided as a file entitled CHMC.P0045USC1_SequenceListing.xml, which was created on Nov. 12, 2025, and is 11,540 bytes in size. The information in the electronic Sequence Listing is hereby incorporated by reference in its entirety.

### FIELD OF THE INVENTION

Aspects of the present disclosure relate generally to hematopoietic stem cell compositions and methods of making thereof.

### BACKGROUND

Clinically, hematopoietic stem cell (HSC) transplants derived from bone marrow are common procedures performed more than 40,000 times a year worldwide. These transplants can be done to treat various types of blood cancers, immune disorders, and gene defects in blood cells. HSC transplants can be performed either autologously (using the patient's own cells) or allogeneically (using a human leukocyte antigen (HLA) matched donor). However, autologous transplants are often not an option due to limited availability of the patient's own healthy cells and allogeneic transplants can result in graft vs. host disease (GVHD) that can be chronic and require immunosuppressants that put the patient at risk for infection. Because of this, allogeneic transplants have a lower survival rate.

While HSCs from umbilical cord blood (UCB) may be collected for use in a patient, the number of cells that can be obtained this way is limited and there are currently no culture methods to significantly expand their numbers in vitro. Furthermore, previous attempts to produce HSCs from iPSCs require introduction of foreign transgenes that pose unknown risks, thus hampering potential clinical applications. There is a present and lasting need for robust production and expansion of HSCs, for example for use in autologous or allogeneic transplants.

### SUMMARY

Some aspects of the present disclosure relate to methods of producing a liver organoid that produces hematopoietic stem cells. In some embodiments, the methods comprise culturing a foregut spheroid under conditions sufficient to differentiate the foregut spheroid into a liver organoid. In some embodiments, the liver organoid comprises a CD34 hemogenic endothelium population that differentiate into hematopoietic stem cells. In some embodiments, culturing the foregut spheroid under conditions sufficient to differentiate the foregut spheroid into a liver organoid comprises contacting the foregut spheroid with one or more (e.g. at least 1, 2, 3) of an FGF signaling pathway activator, a Wnt signaling pathway activator, or a BMP inhibitor, or any combination thereof. In some embodiments, culturing the foregut spheroid under conditions sufficient to differentiate the foregut spheroid into a liver organoid comprises contacting the foregut spheroid with an FGF signaling pathway activator or a Wnt signaling pathway activator, or both. In some embodiments, the FGF signaling pathway activator is FGF4. In some embodiments, the Wnt signaling pathway activator is CHIR99021. In some embodiments, the BMP inhibitor is Noggin. In some embodiments, culturing the foregut spheroid comprises contacting the foregut spheroid with a Notch activator or ligand. In some embodiments, the Notch activator or ligand is, comprises, consists essentially of, or consists of one or more of DLL1, DLL3, DLL4, JAG1, or JAG2, or any combination thereof. In some embodiments, the Notch activator or ligand is, comprises, consists essentially of, or consists of a cell that expresses one or more of DLL1, DLL3, DLL4, JAG1, or JAG2, or any combination thereof. In some embodiments, the foregut spheroid comprises a cell that expresses one or more Notch activators or ligands selected from the group consisting of DLL1, DLL3, DLL4, JAG1, or JAG2, or any combination thereof. In some embodiments, culturing the foregut spheroid comprises contacting the foregut spheroid with one or more of IL-3, IL-34, or GM-CSF, or any combination thereof. In some embodiments, culturing the foregut spheroid comprises contacting the foregut spheroid with stem cell factor (SCF) or thrombopoietin (TPO), or both. In some embodiments, culturing the foregut spheroid does not comprise contacting the foregut spheroid with retinoic acid (RA). In some embodiments, culturing the foregut spheroid does not comprise contacting the foregut spheroid with one or more of dexamethasone, Oncostatin M, or hepatocyte growth factor, or any combination thereof. In some embodiments, the hematopoietic stem cells are not exogenously expressing one or more of ERG, HOXA5, HOXA9, HOXA10, LCOR, RUNX1, SPI1, FOSB, or GFI1, or any combination thereof. In some embodiments, the methods comprise isolating the hemogenic endothelium population or the hematopoietic stem cells, or both, from the liver organoid. In some embodiments, isolating the hemogenic endothelium population or the hematopoietic stem cells, or both, is performed by sorting for a cell population from the liver organoid that is CD34+, CD45+, or both. In some embodiments, the methods comprise culturing induced pluripotent stem cells under conditions sufficient to differentiate the induced pluripotent stem cells into definitive endoderm and culturing the definitive endoderm under conditions sufficient to differentiate the definitive endoderm into the foregut spheroid prior to culturing the foregut spheroid under conditions sufficient to differentiate the foregut spheroid into the liver organoid. In some embodiments, the methods comprise culturing a definitive endoderm under conditions sufficient to differentiate the definitive endoderm into the foregut spheroid prior to culturing the foregut spheroid under conditions sufficient to differentiate the foregut spheroid into the liver organoid.

Some aspects of the present disclosure relate to liver organoids. In some embodiments, the liver organoids are the liver organoids produced by any one of the methods disclosed herein.

Some aspects of the present disclosure relate to a hemogenic endothelium population. In some embodiments, the hemogenic endothelium population is produced by any one of the methods disclosed herein.

Some aspects of the present disclosure relate to hematopoietic stem cells. In some embodiments, the hematopoietic stem cells are produced by any one of the methods disclosed herein.

Some aspects of the present disclosure relate to methods for isolating lymphoid cells from a liver organoid. In some embodiments, the liver organoid is any one of the liver organoids produced by any one of the methods disclosed herein. In some embodiments, the lymphoid cells are isolated from any one of the liver organoids produced by any one of the methods described herein. In some embodiments, the lymphoid cells comprise one or more of B cells, T cells, NK cells, or common lymphoid progenitor cells. In some embodiments, the B cells are B1 cells. In some embodiments, the B cells are B2 cells. In some embodiments, the B cells are both B1 cells and B2 cells. In some embodiments, prior to isolating the lymphoid cells, the methods comprise making a single cell suspension from the liver organoid and culturing the single cell suspension on stromal cells. In some embodiments, culturing the single cell suspension on stromal cells expands lymphoid cells in the single cell suspension. In some embodiments, the stromal cells are MS-5 cells. In some embodiments, the single cell suspension is contacted with one or more of FLT3 ligand, SCF, or IL-7, or any combination thereof. In some embodiments, the B1 cells are CD20⁺CD43⁺CD27⁺. In some embodiments, isolating the B1 cells is performed by sorting for a cell population from the single cell suspension that is one or more of CD20⁺, CD43⁺, CD27⁺, or any combination thereof.

Some aspects of the present disclosure relate to lymphoid cells. In some embodiments, the lymphoid cells are the lymphoid cells produced by any one of the methods described herein. In some embodiments, the lymphoid cells are B1 cells, and the B1 cells are the B1 cells produced by any one of the methods described herein.

Some aspects of the present disclosure relate to methods of expanding a population of hematopoietic stem cells. In some embodiments, the methods comprise culturing the population of hematopoietic stem cells in contact with a liver organoid. In some embodiments, the liver organoid comprises a hematopoietic niche environment. In some embodiments, the liver organoid is the liver organoid produced by any one of the methods described herein. In some embodiments, culturing the population of hematopoietic stem cells comprises contacting the population of hematopoietic stem cells with SCF or TPO, or both. In some embodiments, the expanded population of hematopoietic stem cells maintain pluripotency. In some embodiments, the population of hematopoietic stem cells is obtained from umbilical cord blood, mobilized peripheral blood, bone marrow, pluripotent stem cells, embryonic stem cells, fetal liver, or fetal spleen, or any combination thereof. In some embodiments, the population of hematopoietic stem cells is autologous or allogeneic to the liver organoid. In some embodiments, the population of hematopoietic stem cells is obtained from an individual. In some embodiments, the individual is an individual in need of hematopoietic stem cells. In some embodiments, the liver organoid is produced from induced pluripotent stem cells derived from the individual.

Some aspects of the present disclosure relate to methods of administering to an individual in need a population of hematopoietic stem cells. In some embodiments, the population of hematopoietic stem cells is the population of hematopoietic stem cells produced by any one of the methods described herein. In some embodiments, the population of hematopoietic stem cells is autologous or allogeneic to the individual in need.

Some aspects of the present disclosure relate to a cell composition. In some embodiments, the cell composition comprises a population of hematopoietic stem cells and a liver organoid. In some embodiments, the population of hematopoietic stem cells is the population of hematopoietic stem cells produced by any one of the methods described herein. In some embodiments, the liver organoid is the liver organoid produced by any one of the methods described herein. In some embodiments, the population of hematopoietic stem cells and liver organoid are from the same individual. In some embodiments, the population of hematopoietic stem cells and liver organoid are from different individuals.

Some aspects of the present disclosure relate to methods of treating an individual in need. In some embodiments, the methods comprise administering to the individual in need an isolated hemogenic endothelium population. In some embodiments, the isolated hemogenic endothelium population is the isolated hemogenic endothelium population produced by any one of the methods described herein. In some embodiments, the methods comprise administering to the individual in need isolated hematopoietic stem cells. In some embodiments, the isolated hematopoietic stem cells are the isolated hematopoietic stem cells produced by any one of the methods described herein. In some embodiments, the methods comprise administering to the individual in need isolated lymphoid cells. In some embodiments, the isolated lymphoid cells are the isolated lymphoid cells are produced by any one of the methods described herein. In some embodiments, the methods comprise administering to the individual in need isolated B1 cells. In some embodiments, the isolated B1 cells are the isolated B1 cells produced by any one of the methods described herein.

Embodiments of the present disclosure provided herein are described by way of the following numbered alternatives:

1. A method for generating hematopoietic cells in a liver organoid, comprising contacting a foregut spheroid an FGF signaling pathway activator, a Wnt signaling pathway activator, and a BMP inhibitor to form a liver organoid having a mesoderm cell population; wherein said liver organoid produces hematopoietic stem cells (HSC).

2. The method of alternative 1, wherein said foregut spheroid comprises mesenchyme.

3. The method of alternative 1 or 2, comprising contacting said foregut spheroid with a Notch activator.

4. The method of alternative 3, wherein said Notch activator is selected from one or more proteins selected from DLL1, DLL3, DLL4, JAG1, JAG2, and combinations thereof.

5. The method of alternative 3, wherein said Notch activator is selected from one or more cells expressing DLL1, DLL3, DLL4, JAG1, JAG2, and combinations thereof.

6. The method of any preceding alternative, wherein said Notch activator is a bone marrow cell line expressing one or more of DLL1, DLL3, DLL4, JAG1, JAG2, and combinations thereof.

7. The method of any preceding alternative, comprising the step of contacting said liver organoid with a cytokine.

8. The method of alternative 7, wherein said cytokine is added at a concentration of about 1 to about 10 ng/ml, from about 2 to from about 15 ng/ml, from about 3 to from about 20 ng/ml, from about 4 to from about 25 ng/mL, or from about 5 to from about 30 ng/ml.

9. The method of alternative 1, wherein said HLO expresses one or more of albumin (gene accession number HGNC: 399), alpha-fetoprotein (HGNC: 317), and erythropoietin (HGNC: 3415).

10. The method of any preceding alternatives, wherein said hematopoietic stem cells (HSC) are identified by flow cytometry as one or more of lin−, CD34+, CD38−, CD90+, CD45RA−, CD49f+.

11. The method of any preceding alternative, wherein said hematopoietic stem cells are at a concentration of about 1/10,000 cells in HLO culture.

12. The method of alternative 3, wherein said Notch activator activates Notch 4 (HGNC: 7884).

13. A method for expanding a population of stem cells, comprising culturing said stem cells in the presence of a liver organoid.

14. The method of alternative 13, wherein said stem cells are obtained from umbilical cord blood, peripheral blood, fetal liver, fetal spleen, bone marrow, gut, yolk sac, or combinations thereof.

15. The method of alternative 13 or 14, wherein said expanded stem cells maintain pluripotency.

16. The method of any preceding alternative wherein said stem cells are obtained from an individual in need of treatment with stem cells.

17. The method of any preceding alternative comprising administering said expanded stem cells to an individual in need thereof.

18. The method of any preceding alternative comprising genetically modifying said stem cells one or more time periods selected from before expansion, during expansion, and after expansion.

### BRIEF DESCRIPTION OF THE DRAWINGS

In addition to the features described above, additional features and variations will be readily apparent from the following descriptions of the drawings and exemplary embodiments. It is to be understood that these drawings depict embodiments and are not intended to be limiting in scope.

FIG. 1A depicts an embodiment of a schematic for liver organoid differentiation methods.

FIG. 1B depicts an embodiment of histology of day 18 liver organoid cultures in Matrigel drops with hematoxylin/eosin and immunohistochemistry (IHC) for albumin, alpha-fetoprotein, CD34 and CD45. Boxed regions are shown as zoomed images in bottom right inset.

FIG. 1C depicts an embodiment of expression of CD34, AFP, HBG1, and ALB in liver organoid cultures of different days as measured by rt-PCR.

FIG. 1D depicts an embodiment of albumin expression by liver organoid cultures cultured with liver differentiation media (RA/HCM) or undifferentiated liver organoid (+FGF+BMP/HCM and +FGF+BMP) cultures from different iPSC lines (72_3, 1383D6, 449) as measured by ELISA.

FIG. 2 depicts an embodiment of a t-SNE plot of scRNA-seq data from day 14 organoid cultures with different populations of interest labeled.

FIG. 3 depicts an embodiment of a t-SNE plot of scRNA-seq data from day 14 organoid cultures with liver, endothelial/HSC, and erythroid populations labeled.

FIG. 4A-FIG. 4C depict an embodiment of t-SNE plots of relevant genes indicating (A) a hepatic population, (B) a hemogenic endothelial population, and (C) an erythroid population.

FIG. 5A depicts an embodiment of representative images from Giemsa stains after CFC assays performed from cells of day 14 organoid cultures.

FIG. 5B depicts an embodiment of an assay of colony forming units from CD34⁺ UCB cells, day 18 liver organoid cells, and undifferentiated iPSCs.

FIG. 6A depicts an embodiment of immunofluorescence images showing the presence of liver organoid cells stained for EpCAM, endothelial cells stained for CD34, and an adjacent cluster of erythrocytes stained for GYPA.

FIG. 6B depicts an embodiment of IHC staining of CD34 and CD45 in day 18 organoid cultures.

FIG. 6C depicts an embodiment of flow cytometry plots detecting CD34 in organoid cultures at timepoints day 11, 13, and 15.

FIG. 6D depicts an embodiment of a graph showing number of colonies formed from cells of organoid cultures of different days of differentiation compared to UCB CD34⁺ control.

FIG. 6E depicts an embodiment of a graph showing qPCR results indicating expression levels of HES1 at different days of organoid culture.

FIG. 7A depicts an embodiment of t-SNE plots of scRNA-seq data showing a population of cells co-expressing CD34 and several genes related to Notch signaling.

FIG. 7B depicts an embodiment of a schematic for preparing liver organoids and addition of DLL4 at day 6 of culture.

FIG. 8 depicts an embodiment of CD45 expression in liver organoid cultures after addition of DLL4 and cytokines (IL-3, IL-34, GM-CSF).

FIG. 9A depicts an embodiment of a graph showing qPCR results of IL-6 expression in organoids at day 18 (N=negative control, D=DLL4 added, C=cytokines added, DC=DLL4 and cytokines added).

FIG. 9B depicts an embodiment of a graph showing qPCR results of albumin expression in organoids at day 18 (N=negative control, D=DLL4 added, C-cytokines added, DC=DLL4 and cytokines added).

FIG. 10A depicts an embodiment of light microscopy images of organoid cultures with either DMSO control or 100 μM DAPT after 12 days of treatment.

FIG. 10B depicts an embodiment of a schematic for adding DAPT at day 6 of culture for 4 days (removing DAPT at day 10 of culture).

FIG. 10C depicts an embodiment of HES1 expression at different concentrations of DAPT after 4 days of treatment.

FIG. 10D depicts an embodiment of ALB expression at different concentrations of DAPT after 4 days of treatment.

FIG. 11A depicts an embodiment of a schematic demonstrating a co-culture system layout. MS-5 cells are cultured with FLT3L, SCF, and IL-7.

FIG. 11B depicts an embodiment of brightfield images showing day 20 of MS-5 co-culture with either UCB cells or organoid culture cells.

FIG. 11C depicts an embodiment of flow cytometry plots of day 26 organoid/MS-5 co-cultures. Leftmost panels indicate CD45 and CD20 subsets that are further gated into CD43 and CD27 in the next panels.

FIG. 12A depicts an embodiment of histology of day 18 organoid cultures showing IHC for CD34 and Nestin.

FIG. 12B depicts an embodiment of immunofluorescence images showing DAPI, CD34, and Nestin in day 18 organoid cultures.

FIG. 12C depicts an embodiment of t-SNE plots of scRNA-seq data showing expression of TPO and SCF.

FIG. 12D depicts an embodiment of flow cytometry plots of CD34 and CD45 in day 18 organoid cultures with and without the addition of SCF and TPO.

FIG. 12E depicts an embodiment of a graph showing the number of CFC colonies produced per Matrigel drop at organoid culture day 18 after the addition of UCB cells, SCF, and TPO at organoid culture day 6. “H+U” indicates HLO and UCB cells combined in the Matrigel drop.

FIG. 12F depicts an embodiment of a graph showing the number of CFC colonies formed per 1000 UCB cells. X axis indicates organoid culture day. UCB cells were combined with HLO at organoid culture day 6. “UCB” indicates fresh UCB cell control not co-cultured with HLO. “DMEM-RA” indicates UCB cells and HLO co-cultured in advanced DMEM alone (without retinoic acid). “HCM” indicates UCB cells and HLO co-cultured in 4 days of DMEM+RA (organoid culture days 6-10) followed by HCM liver differentiation media.

FIG. 12G depicts an embodiment of a graph showing relative amounts of different colony types in CFC assays at different conditions shown in FIG. 12F.

FIG. 13A depicts an embodiment of a schematic demonstrating layout of mouse engraftment experiments.

FIG. 13B depicts an embodiment of flow cytometry blots detecting CD45 in isolated PMBCs from mice 7 weeks after injecting cells.

### DETAILED DESCRIPTION

Creation of hematopoietic stem cells (HSCs) from induced pluripotent stem cells (iPSCs) has great potential for clinical use yet has so far been elusive. Recent successes have been seen with the addition of exogenous genes (e.g. ERG, HOXA5, HOXA9, HOXA10, LCOR, RUNX1, SPI1, FOSB, or GFI1) either to iPS cells or endothelial cells for direct conversion. However, the genomic manipulations required for this feat limit any potential clinical translation. At the same time, the advent of organoid culture systems has recently provided researchers with a powerful tool to mimic complicated systems consisting of multiple cell types and study them in a controlled setting over time. The organoid systems disclosed herein recreates the complex niche of the fetal liver microenvironment to provide a more efficient way of studying hematopoiesis in vitro with the goal of creating cells for use in the clinic. Production of hematopoietic cells in organoid systems are explored in PCT Publication WO 2020/056158.

Hematopoiesis in the fetal liver is a unique time window in which HSCs undergo massive expansion. Because of this, it is particularly attractive as an HSC niche to model. HSCs are unable to maintain in culture and have thus far shown limited expansion in vitro, although some recent success has been seen with mouse HSC using a cocktail of growth factors. The current state of the art for in vitro HSC expansion implements a system of media containing hematopoietic cytokines to maintain and expand cells. However, this does not fully reproduce the complex microenvironment involving multiple cell types interacting directly and through transient signaling. The organoid systems described herein provide a unique opportunity to model this niche in a way that allows for expansion of HSCs for therapeutic use.

Modeling the fetal liver microenvironment allows for creation of B1 cells, a unique type of hematopoietic cell that differentiates during fetal development. These cells are made specifically in the fetal liver and exist in only very low levels after birth in the spleen and peritoneum. Due to their exceedingly small quantity in adults, most B1 cell studies use mouse cells while B1 cells in humans have not yet been fully described and potential progenitor populations are still unknown. Another aspect of B1 cells is their ability to secrete IgM without being stimulated. Because of this, they are responsible for the initial response during vaccination. Additional analysis into their differentiation and development may allow for their creation in vitro and provide a useful source of antibodies and aid in potential vaccine development.

The organoid systems described herein are positioned for great clinical impact. Cells from patients needing HSC transplants can have iPSCs generated that are then used to create customized HSCs that can be re-implanted without risk of rejection or GVHD, thus fulfilling the need for a large supply of HSCs for use in autologous or allogeneic transplants.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The embodiments herein are generally disclosed using affirmative language to describe the numerous embodiments. Embodiments also include ones in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about, 80%, 85%, 90%, 91%, 92%, 93%, 94%, 95%, 96%, 97%, 98%, 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between.

Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

Some embodiments described herein relate to pharmaceutical compositions that comprise, consist essentially of, or consist of an effective amount of a cell composition described herein and a pharmaceutically acceptable carrier, excipient, or combination thereof. A pharmaceutical composition described herein is suitable for human and/or veterinary applications.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Soxl, Sox2, Sox3, and Sox15); certain members of the Klf family (e.g., Klfl, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

Blood cell production derives from a single type of cell, the hematopoietic stem cell, which through proliferation and differentiation, gives rise to the entire hematopoietic system. The hematopoietic stem cells are believed to be capable of self-renewal, expanding their own population of stem cells, and they are pluripotent (capable of differentiating into any cell in the hematopoietic system). From this rare cell population, the entire mature hematopoietic system, comprising lymphocytes (B and T cells of the immune system) and myeloid cells (erythrocytes, megakaryocytes, granulocytes and macrophages) is formed. The lymphoid lineage, comprising B cells and T cells, provides for the production of antibodies, regulation of the cellular immune system, detection of foreign agents in the blood, detection of cells foreign to the host, and the like. The myeloid lineage, which includes monocytes, granulocytes, megakaryocytes as well as other cells, monitors for the presence of foreign bodies, provides protection against neoplastic cells, scavenges foreign materials, produces platelets, and the like. The erythroid lineage provides red blood cells, which act as oxygen carriers.

The liver is a vital organ that provides many essential metabolic functions for life such as the detoxification of exogenous compounds and coagulation as well as producing lipids, proteins, ammonium, and bile. Primary hepatocytes are a highly polarized metabolic cell type, and form a bile canaliculi structure with micro villi-lined channels, separating peripheral circulation from the bile acid secretion pathway. In vitro reconstitution of a patient's liver may provide applications including regenerative therapy, drug discovery and drug toxicity studies. Existing methodology using primary liver cells exhibit extremely poor functionality, largely due to a lack of essential anatomical structures, which limits their practical use for the pharmaceutical industry. The formation of liver organoids, which comprise a luminal structure with internalized microvilli and mesenchymal cells, as well as exhibit liver cell types such as hepatocytes, stellate cells, Kupffer cells, and liver endothelial cells, and methods of making and use thereof have previously been described in PCT Publications WO2018/085615, WO2018/085622, WO2018/085623, and WO2018/226267, each of which is hereby expressly incorporated by reference in its entirety.

In some embodiments, ESCs, germ cells, or iPSCs are cultured in growth media that supports the growth of stem cells. In some embodiments, the ESCs, germ cells, or iPSCs are cultured in stem cell growth media. In some embodiments, the stem cell growth media is RPMI 1640, DMEM, DMEM/F12, Advanced DMEM, hepatocyte culture medium (HCM), StemFit, mTeSR 1, or mTeSR Plus media. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors. In some embodiments, the stem cells are grown on a feeder cell substrate. In some embodiments, the stem cells are not grown on a feeder cell substrate. In some embodiments, the stem cells are grown on plates coated with laminin. In some embodiments, the stem cells are grown supplemented with FGF2 or a ROCK inhibitor (e.g. Y-27632), or both. In some embodiments, the FGF2 is at a concentration that is, is about, is at least, is at least about, is not more than, or is not more than about, 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, or 500 ng/mL, or any concentration within a range defined by any two of the aforementioned concentrations, for example, 1 to 500 ng/ml, 10 to 200 ng/ml, 100 to 150 ng/mL, 1 to 100 ng/mL, or 100 to 500 ng/mL. In some embodiments, the ROCK inhibitor is at a concentration that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nM, or any concentration within a range defined by any two of the aforementioned concentrations, for example, 1 to 30 nM, 5 to 25 nM, 10 to 20 nM, 1 to 15 nM, or 10 to 30 nM. In some embodiments, the stem cells are grown on plates coated with Laminin.

### Methods of Definitive Endoderm Differentiation

Any methods for producing definitive endoderm (DE) from pluripotent cells (e.g., iPSCs or ESCs) are applicable to the methods described herein. Exemplary methods are disclosed in, for example, U.S. Pat. No. 9,719,068. In some embodiments, iPSCs are used to produce definitive endoderm.

In some embodiments, one or more growth factors are used in the differentiation process from pluripotent stem cells to DE cells. In some embodiments, the one or more growth factors used in the differentiation process include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, or any combination thereof. In some embodiments, the PSCs are contacted with the one or more growth factors for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 50, 60, 70, 80, 90, 100, 110, 120, 130, 140, 150, 160, 170, 180, 190, 200, 210, 220, 230, or 240 hours, or any number of hours within a range defined by any two of the aforementioned number of days, for example, 1 to 240 hours, 20 to 120 hours, 30 to 50 hours, 1 to 100 hours, or 50 to 240 hours. In some embodiments, the PSCs are contacted with the one or more growth factors at a concentration that is, is about, is at least, is at least about, is not more than, or is not more than about, 10, 20, 30, 40, 50, 60, 70, 80, 90, 100, 200, 300, 400, 500, 600, 700, 800, 900, or 1000 ng/mL, or any concentration within a range defined by any two of the aforementioned concentrations, for example, 10 to 1000 ng/ml, 50 to 800 ng/ml, 100 to 500 ng/mL, 10 to 200 ng/ml or 100 to 1000 ng/mL. In some embodiments, the concentration of the one or more growth factors is maintained at a constant level through the period of contacting. In some embodiments, the concentration of the one or more growth factors is varied during the period of contacting. In some embodiments, the one or more growth factors is dissolved into the growth media. In some embodiments, populations of cells enriched in definitive endoderm cells are used. In some embodiments, the definitive endoderm cells are isolated or substantially purified. In some embodiments, the isolated or substantially purified definitive endoderm cells express one or more (e.g. at least 1, 3) of SOX17, FOXA2, or CXRC4 markers to a greater extent than one or more (e.g. at least 1, 3, 5) of OCT4, AFP, TM, SPARC, or SOX7 markers.

In some embodiments, the definitive endoderm cells are contacted with one or more modulators of a signaling pathway described herein. In some embodiments, the definitive endoderm cells are treated with the one or more modulators of a signaling pathway for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, hours, or 1, 2, 3, 4, 5, 6, 7, 8, 9, 10, 11, 12, 13, 14, 15, 16, 17, 18, 19, or 20 days, or any number of hours or days within a range defined by any two of the aforementioned number of days or hours, for example, 1 hour to 20 days, 20 hours to 10 days, 1 hour to 48 hours, 1 day to 20 days, 1 hour to 5 days, or 24 hours to 20 days. In some embodiments, the concentration of the one or more modulators of a signaling pathway is maintained at a constant level through the period of contacting. In some embodiments, the concentration of the one or more modulators of a signaling pathway is varied during the period of contacting.

In some embodiments, to differentiate the definitive endoderm into foregut spheroids, the definitive endoderm cells are contacted with one or more modulators of an FGF pathway and a Wnt pathway. In some embodiments, cellular constituents associated with the Wnt and/or FGF signaling pathways, for example, natural inhibitors, antagonists, activators, or agonists of the pathways can be used to result in inhibition or activation of the Wnt and/or FGF signaling pathways. In some embodiments, siRNA and/or shRNA targeting cellular constituents associated with the Wnt and/or FGF signaling pathways are used to inhibit or activate these pathways.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. FGFs are key players in the processes of proliferation and differentiation of wide variety of cells and tissues. In humans, 22 members of the FGF family have been identified, all of which are structurally related signaling molecules. Members FGF1 through FGF10 all bind fibroblast growth factor receptors (FGFRs). FGF1 is also known as acidic, and FGF2 is also known as basic fibroblast growth factor (bFGF). Members FGF11, FGF12, FGF13, and FGF14, also known as FGF homologous factors 1˜4 (FHF1-FHF4), have been shown to have distinct functional differences compared to the FGFs. Although these factors possess remarkably similar sequence homology, they do not bind FGFRs and are involved in intracellular processes unrelated to the FGFs. This group is also known as “iFGF.” Members FGF15 through FGF23 are newer and not as well characterized. FGF15 is the mouse ortholog of human FGF19 (hence there is no human FGF15). Human FGF20 was identified based on its homology to Xenopus FGF-20 (XFGF-20). In contrast to the local activity of the other FGFs, FGF15/FGF19, FGF21 and FGF23 have more systemic effects. In some embodiments, the FGF used is one or more (e.g. at least 1, 3, 5) of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, FGF23. In some embodiments, the FGF used is FGF4. In some embodiments, the definitive endoderm is contacted with an FGF at a concentration that is, is about, is at least, is at least about, is not more than, or is not more than about, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 ng/mL, or any concentration within a range defined by any two of the aforementioned concentrations, for example, 10 to 2000 ng/mL, 50 to 1500 ng/mL, 500 to 100 ng/mL, 10 to 1000 ng/ml or 500 to 2000 ng/mL.

In some embodiments, to differentiate the definitive endoderm into foregut spheroids, the definitive endoderm is contacted with a Wnt protein or activator. In some embodiments, the definitive endoderm is contacted with a glycogen synthase kinase 3 (GSK3) inhibitor. GSK3 inhibitor act to activate Wnt pathways. In some embodiments, the definitive endoderm is contacted with the GSK3 inhibitor Chiron (CHIR99021). In some embodiments, the definitive endoderm is contacted with CHIR99021 at a concentration that is, is about, is at least, is at least about, is not more than, or is not more than about, 0.1, 0.2, 0.3, 0.4, 0.5, 0.6, 0.7, 0.8, 0.9, 1, 2, 3, 4, 5, 6, 7, 8, 9, or 10 μM of CHIR99021 or any concentration within a range defined by any two of the aforementioned concentrations, for example, 0.1 to 10 μM, 0.4 to 6 μM, 1 to 5 μM, 0.1 to 1 μM, or 0.5 to 10 UM of CHIR99021.

In some embodiments, the foregut spheroids comprise a mesoderm. In some embodiments, the foregut spheroids comprise mesoderm cells. In some embodiments, the foregut spheroids comprise mesodermal progenitor cells. In some embodiments, the foregut spheroids comprise mesenchyme. In some embodiments, the foregut spheroids comprise mesenchymal cells. In some embodiments, the foregut spheroids comprise mesenchymal stem cells. In some embodiments, the foregut spheroids comprise mesenchymal progenitor cells. In some embodiments, one or more (e.g. at least 1, 2, 3, 4, 5) of the mesoderm, mesoderm cells, mesodermal progenitor cells, mesenchyme, mesenchymal cells, mesenchymal stem cells, or mesenchymal progenitor cells, or any combination thereof, differentiate to a hemogenic endothelium population. In some embodiments, the mesenchyme differentiates into a hemogenic endothelium population. In some embodiments, the hemogenic endothelium population is a CD34 hemogenic endothelium population. In some embodiments, the hemogenic endothelium population is the hemogenic endothelium population of any one of the liver organoids described herein.

### Methods of Foregut Spheroid Differentiation

In some embodiments, the foregut spheroids are differentiated into liver organoids. In some embodiments, the foregut spheroids are differentiated into liver organoids by contacting the foregut spheroids with retinoic acid (RA). In some embodiments, the liver organoids produced from contacting the foregut spheroids resemble mature liver tissue. In some embodiments, the foregut spheroids are differentiated into liver organoids that more closely resemble fetal liver tissue. In some embodiments, the foregut spheroids are differentiated into liver organoids that more closely resemble fetal liver tissue by not contacting the foregut spheroids with RA.

In some embodiments, fetal liver organoids are produced by a Matrigel drop method. In some embodiments, foregut spheroids are embedded in 100% Matrigel and incubated at 37° C. to solidify the Matrigel. In some embodiments, the foregut spheroids embedded in Matrigel are grown in media that does not comprise retinoic acid (e.g. DMEM/F12), for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or any number of days within a range defined by any two of the aforementioned number of days, for example, 1 to 30 days, 10 to 20 days, 12 to 18 days, 1 to 20 days, or 10 to 30 days. In some embodiments, the foregut spheroids are grown in media that does not comprise one or more of dexamethasone, Oncostatin M, or hepatocyte growth factor, or any combination thereof.

In some embodiments, the foregut spheroids or liver organoids, or both, are contacted with a cytokine. In some embodiments, the cytokine is transferring, stem cell factor (SCF), interleukin 3 (IL-3), interleukin 6 (IL-6), interleukin 34 (IL-34), erythropoietin (EPO), granulocyte colony-stimulating factor (G-CSF), granulocyte-macrophage colony-stimulating factor (GM-CSF), or any combination thereof. In some embodiments, the foregut spheroids or liver organoids, or both, are dissociated into single cells before contacting with the cytokine. In some embodiments, the cytokine is at a concentration that is, is about, is at least, is at least about, is not more than, or is not more than about, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000 ng/ml, or 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000 μg/mL, or any concentration within a range defined by any two of the aforementioned concentrations, for example, 1 ng/ml to 1000 μg/mL, 50 ng/ml to 100 μg/mL, 500 ng/ml to 10 μg/mL, 1 ng/mL to 1000 ng/ml, 1 μg/mL to 1000 μg/mL, 1 ng/ml to 50 μg/mL, or 100 ng/ml to 1000 μg/mL. In some embodiments, the foregut spheroids or liver organoids, or both, are contacted with the cytokine for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days within a range defined by any two of the aforementioned number of days, for example, 1 to 50 days, 5 to 40 days, 10 to 30 days, 1 to 30 days, or 20 to 50 days.

In some embodiments, the foregut spheroids or liver organoids, or both, are contacted with a Notch activator or ligand. Notch signaling is important in establishing definitive hematopoiesis in hemogenic endothelium, and a Notch activator or ligand activates the Notch signaling pathway. In some embodiments, a Notch ligand is a Notch activator. In some embodiments, contact with the Notch activator or ligand increases the production of CD45′ hematopoietic cells in the foregut spheroids, or liver organoids, or both. In some embodiments, contact with the Notch activator or ligand increases the number of erythrocytes produced by the foregut spheroids, or liver organoids, or both. In some embodiments, contact with the Notch activator or ligand increases expression of IL-6 in the foregut spheroids, or liver organoids, or both. In some embodiments, contact with the Notch activator or ligand increases expression of HES1 in the foregut spheroids, or liver organoids, or both. In some embodiments, the Notch activator or ligand comprises one or more (e.g. at least 1, 2, 3, 4, 5) of DLL1, DLL3, DLL4, JAG1, or JAG2, or any combination thereof. In some embodiments, the Notch activator or ligand comprises a cell expressing one or more (e.g. at least 1, 2, 3, 4, 5) of DLL1, DLL3, DLL4, JAG1, or JAG2, or any combination thereof. In some embodiments, the foregut spheroid comprises a cell that expresses one or more (e.g. at least 1, 2, 3, 4, 5) Notch activators or ligands selected from the group consisting of DLL1, DLL3, DLL4, JAG1, or JAG2, or any combination thereof. In some embodiments, the foregut spheroid expresses one or more (e.g. at least 1, 2, 3, 4, 5) Notch activators selected from the group consisting of DLL1, DLL3, DLL4, JAG1, or JAG2, or any combination thereof. In some embodiments, the Notch activator or ligand is DLL4. In some embodiments, the foregut spheroids or liver organoids, or both, are dissociated into single cells before contacting with the Notch activator or ligand. In some embodiments, the Notch activator or ligand is at a concentration that is, is about, is at least, is at least about, is not more than, or is not more than about, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000 ng/mL, or 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000 μg/mL, or any concentration within a range defined by any two of the aforementioned concentrations, for example, 1 ng/mL to 1000 μg/mL, 50 ng/ml to 100 μg/mL, 500 ng/ml to 10 μg/mL, 1 ng/mL to 1000 ng/ml, 1 μg/mL to 1000 μg/mL, 1 ng/ml to 50 μg/mL, or 100 ng/mL to 1000 μg/mL. In some embodiments, the foregut spheroids or liver organoids, or both, are contacted with the Notch activator or ligand for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days within a range defined by any two of the aforementioned number of days, for example, 1 to 50 days, 5 to 40 days, 10 to 30 days, 1 to 30 days, or 20 to 50 days.

In some embodiments, albumin secretion of the foregut spheroids or liver organoids, or both, is quantified. In some embodiments, albumin secretion is quantified by ELISA. In some embodiments, the foregut spheroids or liver organoids, or both, are examined by immunohistochemistry. In some embodiments, the foregut spheroids, or liver organoids, or both, are examined for albumin, CD34, or CD45 expression, or any combination thereof. In some embodiments, the foregut spheroids, or liver organoids, or both, are examined by flow cytometry. In some embodiments, the foregut spheroids, or liver organoids, or both are examined for CD45, CD34, CD11b, CD19, CD27, CD43, or CD45 expression, or any combination thereof. In some embodiments, the foregut spheroids, or liver organoids, or both are examined with a hematopoietic colony forming cell assay. In some embodiments, the foregut spheroids, or liver organoids, or both, produce erythroid colonies, myeloid colonies, or lymphoid colonies, or any combination thereof. In some embodiments, the foregut spheroids, or liver organoids, or both, are examined by RT-qPCR. In some embodiments, RT-qPCR is performed using the sequences of SEQ ID NO:1-12. In some embodiments, the foregut spheroids, or liver organoids, or both, are examined by RT-qPCR to quantify expression of AFP, ALB, CD34, HBG1, HES1, or HES5, or any combination thereof. In some embodiments, the foregut spheroids, or liver organoids, or both, are examined by single cell RNA sequencing.

In some embodiments, methods of producing a liver organoid that produces hematopoietic stem cells (HSCs) are provided. In some embodiments, the methods comprise culturing induced pluripotent stem cells under conditions sufficient to differentiate the induced pluripotent stem cells into definitive endoderm, culturing the definitive endoderm under conditions sufficient to differentiate the definitive endoderm into a foregut spheroid, and culturing the foregut spheroid under conditions sufficient to differentiate the foregut spheroid into a liver organoid. In some embodiments, the methods comprise culturing induced pluripotent stem cells under conditions sufficient to differentiate the induced pluripotent stem cells into definitive endoderm. In some embodiments, the methods comprise culturing a definitive endoderm under conditions sufficient to differentiate the definitive endoderm into a foregut spheroid. In some embodiments, the methods comprise culturing a foregut spheroid under conditions sufficient to differentiate the foregut spheroid into a liver organoid. In some embodiments, culturing the foregut spheroid under conditions sufficient to differentiate the foregut spheroid into a liver organoid comprises contacting the foregut spheroid with one or more (e.g. at least 1, 2, 3) of an FGF signaling pathway activator, a Wnt signaling pathway activator, or a BMP inhibitor, or any combination thereof. In some embodiments, culturing the foregut spheroid under conditions sufficient to differentiate the foregut spheroid into a liver organoid comprises contacting the foregut spheroid with an FGF signaling pathway activator or a Wnt signaling pathway activator, or both. In some embodiments, the FGF signaling pathway activator is FGF4. In some embodiments, the Wnt signaling pathway activator is CHIR99021. In some embodiments, the BMP inhibitor is Noggin. In some embodiments, the foregut spheroid comprises a mesoderm. In some embodiments, the foregut spheroid comprises mesoderm cells. In some embodiments, the foregut spheroid comprises mesodermal progenitor cells. In some embodiments, the foregut spheroid comprises mesenchyme. In some embodiments, the foregut spheroid comprises mesenchymal cells. In some embodiments, the foregut spheroid comprises mesenchymal stem cells. In some embodiments, the foregut spheroid comprises mesenchymal progenitor cells. In some embodiments, one or more (e.g. at least 1, 2, 3, 4, 5) of the mesoderm, mesoderm cells, mesodermal progenitor cells, mesenchyme, mesenchymal cells, mesenchymal stem cells, mesenchymal stem cells or mesenchymal progenitor cells, or any combination thereof, differentiate to a hemogenic endothelium population. In some embodiments, the hemogenic endothelium population is a CD34⁺ hemogenic endothelium population. In some embodiments, the liver organoid comprises a CD34⁺ hemogenic endothelium population. In some embodiments, the hemogenic endothelium population differentiates into hematopoietic stem cells. In some embodiments, the hemogenic endothelium population of the liver organoid is differentiated from one or more (e.g. at least 1, 2, 3, 4, 5) of the mesoderm, mesoderm cells, mesodermal progenitor cells, mesenchyme, mesenchymal cells, mesenchymal stem cells, or mesenchymal progenitor cells, or any combination thereof, of the foregut spheroid. In some embodiments, the hemogenic endothelium population of the liver organoid is differentiated from the mesenchyme of the foregut spheroid. In some embodiments, the hemogenic endothelium population differentiates into erythrocytes. In some embodiments, the hemogenic endothelium population differentiates into erythrocytes without the addition of exogenous hematopoietic cytokines. In some embodiments, the liver organoid comprises one or more (e.g. at least 1, 2, 3) of a hepatic population, an erythroid population, or a hemogenic endothelium population, or any combination thereof. In some embodiments, the liver organoid comprises cells that express EpCAM. In some embodiments, the liver organoid comprises CD34 endothelium. In some embodiments, the CD34 endothelium expresses one or more (e.g. at least 1, 3, 5) of Notch1, Notch4, HEY1, HEY2, DLL4, or GATA2, or any combination thereof. In some embodiments, the liver organoid comprises erythroid cells expressing GYPA. In some embodiments, the liver organoid comprises CD45-expressing hematopoietic cells.

In some embodiments, the induced pluripotent stem cells are cultured for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days within a range defined by any two of the aforementioned number of days, for example, 1 to 50 days, 5 to 40 days, 10 to 30 days, 1 to 30 days, or 20 to 50 days. In some embodiments, the definitive endoderm is cultured for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days within a range defined by any two of the aforementioned number of days, for example, 1 to 50 days, 5 to 40 days, 10 to 30 days, 1 to 30 days, or 20 to 50 days. In some embodiments, the foregut spheroids are cultured for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days within a range defined by any two of the aforementioned number of days, for example, 1 to 50 days, 5 to 40 days, 10 to 30 days, 1 to 30 days, or 20 to 50 days.

In some embodiments, culturing the induced pluripotent stem cells comprise contacting the induced pluripotent stem cells with one or more (e.g. at least 1 or 2) of Activin A or BMP4. In some embodiments, culturing the definitive endoderm comprises contacting the definitive endoderm with one or more (e.g. at least 1 or 2) of CHIR99021 or FGF. In some embodiments, culturing the foregut spheroid comprises contacting the foregut spheroid with one or more of BMP4 and FGF2. In some embodiments, culturing the foregut spheroid comprises contacting the foregut spheroid with one or more of BMP4 and FGF2 in an extracellular matrix. In some embodiments, culturing the foregut spheroid comprises contacting the foregut spheroid with a Notch activator or ligand. In some embodiments, the Notch activator or ligand comprises one or more (e.g. at least 1, 3, 5) of DLL1, DLL3, DLL4, JAG1, or JAG2, or any combination thereof. In some embodiments, the Notch activator or ligand comprises a cell that expresses one or more (e.g. at least 1, 3, 5) of DLL1, DLL3, DLL4, JAG1, or JAG2, or any combination thereof. In some embodiments, the foregut spheroid comprises a cell that expresses one or more (e.g. at least 1, 2, 3, 4, 5) Notch activators or ligands selected from the group consisting of DLL1, DLL3, DLL4, JAG1, or JAG2, or any combination thereof. In some embodiments, the foregut spheroid expresses one or more (e.g. at least 1, 2, 3, 4, 5) Notch activators or ligands selected from the group consisting of DLL1, DLL3, DLL4, JAG1, or JAG2, or any combination thereof. In some embodiments, the Notch activator or ligand is DLL4. In some embodiments, culturing the foregut spheroid comprises contacting the foregut spheroid with one or more (e.g. at least 1, 2, 3) of IL-3, IL-34, or GM-CSF, or any combination thereof. In some embodiments, culturing the foregut spheroid comprises contacting the foregut spheroid with stem cell factor (SCF) or thrombopoietin (TPO), or both. In some embodiments, culturing the foregut spheroid further comprises contacting the foregut spheroid with one or more (e.g. at least 1, 2, 3, 4, 5) of IL-3, IL-34, EPO, G-CSF, GM-CSF, SCF, or TPO, or any combination thereof. In some embodiments, culturing the foregut spheroid does not comprise contacting the foregut spheroid with retinoic acid (RA). In some embodiments, culturing the foregut spheroid does not comprise contacting the foregut spheroid with one or more (e.g. at least 1, 2, 3 4) of RA, dexamethasone, Oncostatin M, or hepatocyte growth factor, or any combination thereof.

Described herein are methods of producing a liver organoid that produces hematopoietic stem cells. The methods comprise culturing a foregut spheroid under conditions sufficient to differentiate the foregut spheroid into a liver organoid. In some embodiments, the foregut spheroid comprises a mesenchyme. In some embodiments, the liver organoid comprises a CD34 hemogenic endothelium population that differentiate into hematopoietic stem cells. In some embodiments, the CD34 hemogenic endothelium population originates from the mesenchyme of the foregut spheroid. In some embodiments, the foregut spheroid is obtained by culturing PSCs, iPSCs, or definitive endoderm according to any one of the methods described herein. In some embodiments, PSCs or iPSCs are cultured according to any one of the methods described herein for a number of days that is, is about, is at least, is at least about, is not more than, or is not more than about, 1, 2, 3, 4, or 5 days to obtain definitive endoderm. In some embodiments, PSCs or iPSCs are cultured with Activin A or BMP4, or both, for a number of days that is, is about, is at least, is at least about, is not more than, or is not more than about, 1, 2, 3, 4, or 5 days to obtain definitive endoderm. In some embodiments, definitive endoderm is cultured according to any one of the methods described herein for a number of days that is, is about, is at least, is at least about, is not more than, or is not more than about, 1, 2, 3, 4, or 5 days to obtain foregut spheroids. In some embodiments, definitive endoderm is cultured with FGF4 or CHIR99021, or both, for a number of days is, is about, is at least, is at least about, is not more than, or is not more than about, 1, 2, 3, 4, or 5 days to obtain foregut spheroids. In some embodiments, culturing the foregut spheroid comprises contacting the foregut spheroid with a Notch activator or ligand. In some embodiments, the Notch activator or ligand is provided at a concentration that is, is about, is at least, is at least about, is not more than, or is not more than about, 1, 2, 3, 4, 5, 6, 7, 8, 9, 10, 15, 20, 25, 30, 35, 40, 45, or 50 μg/mL, or any concentration within a range defined by any two of the aforementioned concentrations, for example, 1 ng/ml to 50 μg/mL, 5 ng/ml to 40 μg/mL, 10 ng/ml to 30 μg/mL, 1 ng/ml to 20 ng/ml, or 5 μg/mL to 20 μg/mL. In some embodiments, the foregut spheroid is contacted with the Notch activator or ligand for a number of days that is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 14 days, 1 to 15 days, or 5 to 20 days.

Described herein are methods of producing a liver organoid that produces hematopoietic stem cells. The methods comprise culturing a foregut spheroid under conditions sufficient to differentiate the foregut spheroid into a liver organoid. In some embodiments, the foregut spheroid comprises a mesenchyme. In some embodiments, the liver organoid comprises a CD34 hemogenic endothelium population that differentiate into hematopoietic stem cells. In some embodiments, the CD34 hemogenic endothelium population originates from the mesenchyme of the foregut spheroid. In some embodiments, the foregut spheroid is obtained by culturing PSCs, iPSCs, or definitive endoderm according to any one of the methods described herein. In some embodiments, PSCs or iPSCs are cultured according to any one of the methods described herein for a number of days that is, is about, is at least, is at least about, is not more than, or is not more than about, 1, 2, 3, 4, or 5 days to obtain definitive endoderm. In some embodiments, PSCs or iPSCs are cultured with Activin A or BMP4, or both, for a number of days that is, is about, is at least, is at least about, is not more than, or is not more than about, 1, 2, 3, 4, or 5 days to obtain definitive endoderm. In some embodiments, definitive endoderm is cultured according to any one of the methods described herein for a number of days that is, is about, is at least, is at least about, is not more than, or is not more than about, 1, 2, 3, 4, or 5 days to obtain foregut spheroids. In some embodiments, definitive endoderm is cultured with FGF4 or CHIR99021, or both, for a number of days is, is about, is at least, is at least about, is not more than, or is not more than about, 1, 2, 3, 4, or 5 days to obtain foregut spheroids. In some embodiments, culturing the foregut spheroid comprises contacting the foregut spheroid with one or more (e.g. at least 1, 2, 3, 4, 5) of the Notch activators or ligands DLL1, DLL3, DLL4, JAG1, or JAG2, or any combination thereof. In some embodiments, one or more (e.g. at least 1, 2, 3, 4, 5) of the Notch activators or ligands DLL1, DLL3, DLL4, JAG1, or JAG2, or any combination thereof, is provided at a concentration that is, is about, is at least, is at least about, is not more than, or is not more than about, 1, 2, 3, 4, 5, 6, 7, 8, 9, 10, 15, 20, 25, 30, 35, 40, 45, or 50 μg/mL, or any concentration within a range defined by any two of the aforementioned concentrations, for example, 1 ng/ml to 50 μg/mL, 5 ng/ml to 40 μg/mL, 10 ng/ml to 30 μg/mL, 1 ng/ml to 20 ng/ml, or 5 μg/mL to 20 μg/mL. In some embodiments, the foregut spheroid is contacted with one or more (e.g. at least 1, 2, 3, 4, 5) of the Notch activators or ligands DLL1, DLL3, DLL4, JAG1, or JAG2, or any combination thereof, for a number of days that is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 14 days, 1 to 15 days, or 5 to 20 days. In some embodiments, the foregut spheroid comprises a cell that expresses one or more (e.g. at least 1, 2, 3, 4 5) Notch activators or ligands selected from the group consisting of DLL1, DLL3, DLL4, JAG1, or JAG2, or any combination thereof. In some embodiments, the foregut spheroid expresses one or more (e.g. at least 1, 2, 3, 4 5) Notch activators or ligands selected from the group consisting of DLL1, DLL3, DLL4, JAG1, or JAG2, or any combination thereof. In some embodiments, the Notch activator or ligand is DLL4.

Described herein are methods of producing a liver organoid that produces hematopoietic stem cells. The methods comprise culturing a foregut spheroid under conditions sufficient to differentiate the foregut spheroid into a liver organoid. In some embodiments, the foregut spheroid comprises a mesenchyme. In some embodiments, the liver organoid comprises a CD34⁺ hemogenic endothelium population that differentiate into hematopoietic stem cells. In some embodiments, the CD34⁺ hemogenic endothelium population originates from the mesenchyme of the foregut spheroid. In some embodiments, the foregut spheroid is obtained by culturing PSCs, iPSCs, or definitive endoderm according to any one of the methods described herein. In some embodiments, PSCs or iPSCs are cultured according to any one of the methods described herein for a number of days that is, is about, is at least, is at least about, is not more than, or is not more than about, 1, 2, 3, 4, or 5 days to obtain definitive endoderm. In some embodiments, PSCs or iPSCs are cultured with Activin A or BMP4, or both, for a number of days that is, is about, is at least, is at least about, is not more than, or is not more than about, 1, 2, 3, 4, or 5 days to obtain definitive endoderm. In some embodiments, definitive endoderm is cultured according to any one of the methods described herein for a number of days that is, is about, is at least, is at least about, is not more than, or is not more than about, 1, 2, 3, 4, or 5 days to obtain foregut spheroids. In some embodiments, definitive endoderm is cultured with FGF4 or CHIR99021, or both, for a number of days is, is about, is at least, is at least about, is not more than, or is not more than about, 1, 2, 3, 4, or 5 days to obtain foregut spheroids. In some embodiments, culturing the foregut spheroid comprises contacting the foregut spheroid with DLL4. In some embodiments, DLL4 is provided at a concentration that is, is about, is at least, is at least about, is not more than, or is not more than about, 1, 2, 3, 4, 5, 6, 7, 8, 9, 10, 15, 20, 25, 30, 35, 40, 45, or 50 μg/mL, or any concentration within a range defined by any two of the aforementioned concentrations, for example, 1 ng/ml to 50 μg/mL, 5 ng/ml to 40 μg/mL, 10 ng/ml to 30 μg/mL, 1 ng/ml to 20 ng/ml, or 5 μg/mL to 20 μg/mL. In some embodiments, the foregut spheroid is contacted with DLL4 for a number of days that is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 14 days, 1 to 15 days, or 5 to 20 days.

Described herein are methods of producing a liver organoid that produces hematopoietic stem cells. The methods comprise culturing a foregut spheroid under conditions sufficient to differentiate the foregut spheroid into a liver organoid. In some embodiments, the foregut spheroid comprises a mesenchyme. In some embodiments, the liver organoid comprises a CD34 hemogenic endothelium population that differentiate into hematopoietic stem cells. In some embodiments, the CD34 hemogenic endothelium population originates from the mesenchyme of the foregut spheroid. In some embodiments, the foregut spheroid is obtained by culturing PSCs, iPSCs, or definitive endoderm according to any one of the methods described herein. In some embodiments, PSCs or iPSCs are cultured according to any one of the methods described herein for a number of days that is, is about, is at least, is at least about, is not more than, or is not more than about, 1, 2, 3, 4, or 5 days to obtain definitive endoderm. In some embodiments, PSCs or iPSCs are cultured with Activin A or BMP4, or both, for a number of days that is, is about, is at least, is at least about, is not more than, or is not more than about, 1, 2, 3, 4, or 5 days to obtain definitive endoderm. In some embodiments, definitive endoderm is cultured according to any one of the methods described herein for a number of days that is, is about, is at least, is at least about, is not more than, or is not more than about, 1, 2, 3, 4, or 5 days to obtain foregut spheroids. In some embodiments, definitive endoderm is cultured with FGF4 or CHIR99021, or both, for a number of days is, is about, is at least, is at least about, is not more than, or is not more than about, 1, 2, 3, 4, or 5 days to obtain foregut spheroids. In some embodiments, culturing the foregut spheroid comprises contacting the foregut spheroid with a cytokine. In some embodiments, the cytokine is provided at a concentration that is, is about, is at least, is at least about, is not more than, or is not more than about, 0, 10, 20, 30, 40, 50, 60, 70, 80, 90, 100, 110, 120, 130, 140, 150, 160, 170, 180, 190, or 200 ng/mL, or any concentration within a range defined by any two of the aforementioned concentrations, for example, 0 to 200 ng/mL, 50 to 150 ng/mL, 80 to 120 ng/ml, 0 to 100 ng/mL, or 100 to 200 ng/ml. In some embodiments, the cytokine is one or more (e.g. at least 1, 2, 3) of IL-3, IL-34, or GM-CSF, or any combination thereof. In some embodiments, the foregut spheroid is contacted with the Notch activator or ligand for a number of days that is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 14 days, 1 to 15 days, or 5 to 20 days.

Described herein are methods of producing a liver organoid that produces hematopoietic stem cells. The methods comprise culturing a foregut spheroid under conditions sufficient to differentiate the foregut spheroid into a liver organoid. In some embodiments, the foregut spheroid comprises a mesenchyme. In some embodiments, the liver organoid comprises a CD34 hemogenic endothelium population that differentiate into hematopoietic stem cells. In some embodiments, the CD34 hemogenic endothelium population originates from the mesenchyme of the foregut spheroid. In some embodiments, the foregut spheroid is obtained by culturing PSCs, iPSCs, or definitive endoderm according to any one of the methods described herein. In some embodiments, PSCs or iPSCs are cultured according to any one of the methods described herein for a number of days that is, is about, is at least, is at least about, is not more than, or is not more than about, 1, 2, 3, 4, or 5 days to obtain definitive endoderm. In some embodiments, PSCs or iPSCs are cultured with Activin A or BMP4, or both, for a number of days that is, is about, is at least, is at least about, is not more than, or is not more than about, 1, 2, 3, 4, or 5 days to obtain definitive endoderm. In some embodiments, definitive endoderm is cultured according to any one of the methods described herein for a number of days that is, is about, is at least, is at least about, is not more than, or is not more than about, 1, 2, 3, 4, or 5 days to obtain foregut spheroids. In some embodiments, definitive endoderm is cultured with FGF4 or CHIR99021, or both, for a number of days is, is about, is at least, is at least about, is not more than, or is not more than about, 1, 2, 3, 4, or 5 days to obtain foregut spheroids. In some embodiments, culturing the foregut spheroid comprises contacting the foregut spheroid with one or more (e.g. at least 1, 2, 3, 4, 5) of the cytokines SCF, IL-3, IL-6, IL-34, EPO, TPO, G-CSF, or GM-CSF, or any combination thereof. In some embodiments, one or more (e.g. at least 1, 2, 3, 4, 5) of the cytokines SCF, IL-3, IL-6, IL-34, EPO, TPO, G-CSF, or GM-CSF, or any combination thereof, is provided at a concentration that is, is about, is at least, is at least about, is not more than, or is not more than about, 0, 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, or 200 ng/ml, or any concentration within a range defined by any two of the aforementioned concentrations, for example, 0 to 200 ng/ml, 5 to 10 ng/mL, 20 to 100 ng/ml, or 100 to 200 ng/mL. In some embodiments, one or more (e.g., at least 1, 2, 3, 4, 5) of the cytokines SCF, IL-3, IL-6, IL-34, EPO, TPO, G-CSF, or GM-CSF, or any combination thereof, is provided at a concentration that is, is about, is at least, is at least about, is not more than, or is not more than about, 0, 1, 2, 3, 4, 5, 6, 7, 8, 9, 10, 11, 12, 13, 14, 15, 16, 17, 18, 19, 20, 30, 40, 50, 60, 70, 80, 90, 100 μg/mL, or any concentration within a range defined by any two of the aforementioned concentrations, for example, 0 to 100 μg/mL, 1 to 10 μg/mL, 10 to 50 μg/mL, 20 to 100 μg/mL, or 5 to 15 μg/mL. In some embodiments, the foregut spheroid is contacted with one or more (e.g. at least 1, 2, 3, 4, 5) of the cytokines SCF, IL-3, IL-6, IL-34, EPO, TPO, G-CSF, or GM-CSF, or any combination thereof, for a number of days that is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 14 days, 1 to 15 days, or 5 to 20 days.

Described herein are methods of producing a liver organoid that produces hematopoietic stem cells. The methods comprise culturing a foregut spheroid under conditions sufficient to differentiate the foregut spheroid into a liver organoid. In some embodiments, the foregut spheroid comprises a mesenchyme. In some embodiments, the liver organoid comprises a CD34 hemogenic endothelium population that differentiate into hematopoietic stem cells. In some embodiments, the CD34⁺ hemogenic endothelium population originates from the mesenchyme of the foregut spheroid. In some embodiments, the foregut spheroid is obtained by culturing PSCs, iPSCs, or definitive endoderm according to any one of the methods described herein. In some embodiments, PSCs or iPSCs are cultured according to any one of the methods described herein for a number of days that is, is about, is at least, is at least about, is not more than, or is not more than about, 1, 2, 3, 4, or 5 days to obtain definitive endoderm. In some embodiments, PSCs or iPSCs are cultured with Activin A or BMP4, or both, for a number of days that is, is about, is at least, is at least about, is not more than, or is not more than about, 1, 2, 3, 4, or 5 days to obtain definitive endoderm. In some embodiments, definitive endoderm is cultured according to any one of the methods described herein for a number of days that is, is about, is at least, is at least about, is not more than, or is not more than about, 1, 2, 3, 4, or 5 days to obtain foregut spheroids. In some embodiments, definitive endoderm is cultured with FGF4 or CHIR99021, or both, for a number of days is, is about, is at least, is at least about, is not more than, or is not more than about, 1, 2, 3, 4, or 5 days to obtain foregut spheroids. In some embodiments, culturing the foregut spheroid comprises contacting the foregut spheroid with one or more (e.g. at least 1, 2, 3) of IL-3, IL-34, or GM-CSF, or any combination thereof. In some embodiments, one or more (e.g. at least 1, 2, 3) of IL-3, IL-34, or GM-CSF, or any combination thereof, is provided at a concentration that is, is about, is at least, is at least about, is not more than, or is not more than about, 0, 10, 20, 30, 40, 50, 60, 70, 80, 90, 100, 110, 120, 130, 140, 150, 160, 170, 180, 190, or 200 ng/ml, or any concentration within a range defined by any two of the aforementioned concentrations, for example, 0 to 200 ng/mL, 50 to 150 ng/ml, 80 to 120 ng/mL, 0 to 100 ng/mL, or 100 to 200 ng/ml. In some embodiments, the cytokine is one or more (e.g. at least 1, 2, 3) of IL-3, IL-34, or GM-CSF, or any combination thereof. In some embodiments, the foregut spheroid is contacted with one or more (e.g. at least 1, 2, 3) of IL-3, IL-34, or GM-CSF, or any combination thereof, for a number of days that is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 14 days, 1 to 15 days, or 5 to 20 days.

In some embodiments, any one of the methods described herein further comprise culturing induced pluripotent stem cells under conditions sufficient to differentiate the induced pluripotent stem cells into definitive endoderm and culturing the definitive endoderm under conditions sufficient to differentiate the definitive endoderm into the foregut spheroid prior to culturing the foregut spheroid under conditions sufficient to differentiate the foregut spheroid into the liver organoid. In some embodiments, any one of the methods described herein further comprise culturing a definitive endoderm under conditions sufficient to differentiate the definitive endoderm into the foregut spheroid prior to culturing the foregut spheroid under conditions sufficient to differentiate the foregut spheroid into the liver organoid. In some embodiments, the conditions sufficient to differentiate the induced pluripotent stem cells into definitive endoderm comprises contacting the induced pluripotent stem cells with Activin A or BMP4, or both, to obtain the definitive endoderm. In some embodiments, the conditions sufficient to differentiate the definitive endoderm into the foregut spheroid comprises contacting the definitive endoderm with FGF4 or CHIR99021, or both, to obtain the foregut spheroid. In some embodiments, the foregut spheroid comprises mesoderm, mesoderm cells, mesodermal progenitor cells, mesenchyme, mesenchymal cells, mesenchymal stem cells, or mesenchymal progenitor cells, or any combination thereof. In some embodiments, the foregut spheroid comprises mesenchyme. In some embodiments, one or more of the induced pluripotent stem cells, definitive endoderm, foregut spheroids, or liver organoid, or any combination thereof is prepared according to methods described herein. In some embodiments, one or more of the induced pluripotent stem cells, definitive endoderm, foregut spheroids, or liver organoid, or any combination thereof is prepared according to methods described in PCT Publications WO 2018/085615, WO 2018/191673, WO 2018/226267, WO 2019/126626, WO 2020/023245, WO 2020/056158, and WO 2020/069285, each of which is hereby expressly incorporated by reference for the purposes of producing induced pluripotent stem cells, definitive endoderm, foregut spheroids, or liver organoids, or any combination thereof.

As described herein, some embodiments of liver organoids produce one or more (e.g. at least 1, 2, 3) of hemogenic endothelium cells, hematopoietic cells, hematopoietic stem cells, or hematopoietic progenitor cells, or any combination thereof. In some embodiments, the one or more of hemogenic endothelium cells, hematopoietic cells, hematopoietic stem cells, or hematopoietic progenitor cells, or any combination thereof, originate from mesenchyme of the liver organoids. In some embodiments, the one or more of hemogenic endothelium cells, hematopoietic cells, hematopoietic stem cells, or hematopoietic progenitor cells, or any combination thereof, originate from mesenchyme of the foregut spheroids that differentiate into the liver organoids. In some embodiments, the hemogenic endothelium cells, hematopoietic cells, hematopoietic stem cells, or hematopoietic progenitor cells, or any combination thereof, are isolated from the liver organoids. In some embodiments, the hemogenic endothelium cells, hematopoietic cells, hematopoietic stem cells, or hematopoietic progenitor cells, or any combination thereof, that are isolated are administered to an individual in need. In some embodiments, the hemogenic endothelium cells, hematopoietic cells, hematopoietic stem cells, or hematopoietic progenitor cells, or any combination thereof, that are isolated from the liver organoid are allogeneic to the individual. In some embodiments, the liver organoid is autologous to the individual, and as a consequence, the hemogenic endothelium cells, hematopoietic cells, hematopoietic stem cells, or hematopoietic progenitor cells, or any combination thereof, are also autologous to the individual. In some embodiments, the hemogenic endothelium cells, hematopoietic cells, hematopoietic stem cells, or hematopoietic progenitor cells, or any combination thereof, are not exogenously expressing one or more (e.g. at least 1, 3, 5) of ERG, HOXA5, HOXA9, HOXA10, LCOR, RUNX1, SPI1, FOSB, or GFI1, or any combination thereof.

In some embodiments, the methods described herein comprise isolating the hemogenic endothelium population or the hematopoietic stem cells, or both, from the liver organoid. In some embodiments, isolating the hemogenic endothelium population or the hematopoietic stem cells, or both, is performed by sorting for a cell population from the liver organoid that is CD34+, CD45+, or both.

In some embodiments, the methods described herein comprise isolating lymphoid cells from a liver organoid. In some embodiments, the liver organoid is any one of the liver organoids produced by any one of the methods described herein. In some embodiments, the lymphoid cells are isolated from any one of the liver organoids produced by any one of the methods described herein. In some embodiments, the lymphoid cells comprise one or more of B cells, T cells, NK cells, or common lymphoid progenitor cells. In some embodiments, the B cells are B1 cells. In some embodiments, the B cells are B2 cells. In some embodiments, the B cells are both B1 cells and B2 cells. In some embodiments, the lymphoid cells that are isolated are B1 cells. In some embodiments, the methods described herein comprise making a single cell suspension from the liver organoid. In some embodiments, the methods described herein comprise culturing the single cell suspension on stromal cells. In some embodiments, the methods described herein comprise culturing the single cell suspension on stromal cells prior to isolating the lymphoid cells. In some embodiments, culturing the single cell suspension on stromal cells expands the lymphoid cells in the single cell suspension. In some embodiments, the stromal cells are MS-5 cells. In some embodiments, the single cell suspension is cultured on stromal cells for a number of weeks that is, is about, is at least, is at least about, is not more than, or is not more than about, 2, 3, 4, or 5 weeks. In some embodiments, the single cell suspension is contacted with one or more cytokines. In some embodiments, one or more cytokines, or any combination thereof, is provided at a concentration that is, is about, is at least, is at least about, is not more than, or is not more than about, 0, 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, or 200 ng/ml, or any concentration within a range defined by any two of the aforementioned concentrations, for example, 0 to 200 ng/ml, 5 to 10 ng/mL, 20 to 100 ng/mL, or 100 to 200 ng/ml. In some embodiments, the liver organoid is contacted with one or more cytokines for a number of days that is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 14 days, 1 to 15 days, or 5 to 20 days. In some embodiments, the single cell suspension is contacted with one or more (e.g. at least 1, 2, 3) of SCF, IL-3, IL-6, IL-7, IL-34, EPO, TPO, G-CSF, GM-CSF, or Fms-like tyrosine kinase 3 ligand (FLT3 ligand, FLT3L), or any combination thereof. In some embodiments, one or more (e.g. at least 1, 2, 3) of SCF, IL-3, IL-6, IL-7, IL-34, EPO, TPO, G-CSF, GM-CSF, or FLT3 ligand, or any combination thereof, is provided at a concentration that is, is about, is at least, is at least about, is not more than, or is not more than about, 0, 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, or 200 ng/ml, or any concentration within a range defined by any two of the aforementioned concentrations, for example, 0 to 200 ng/ml, 5 to 10 ng/ml, 20 to 100 ng/mL, or 100 to 200 ng/mL. In some embodiments, one or more (e.g. at least 1, 2, 3) of SCF, IL-3, IL-6, IL-7, IL-34, EPO, TPO, G-CSF, GM-CSF, or FLT3 ligand, or any combination thereof, is provided at a concentration that is, is about, is at least, is at least about, is not more than, or is not more than about, 0, 1, 2, 3, 4, 5, 6, 7, 8, 9, 10, 11, 12, 13, 14, 15, 16, 17, 18, 19, 20, 30, 40, 50, 60, 70, 80, 90, 100 μg/mL, or any concentration within a range defined by any two of the aforementioned concentrations, for example, 0 to 100 μg/mL, 1 to 10 μg/mL, 10 to 50 μg/mL, 20 to 100 μg/mL, or 5 to 15 μg/mL. In some embodiments, the liver organoid is contacted with one or more (e.g. at least 1, 2, 3) of SCF, IL-3, IL-6, IL-7, IL-34, EPO, TPO, G-CSF, GM-CSF, or FLT3 ligand, or any combination thereof, for a number of days that is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 14 days, 1 to 15 days, or 5 to 20 days. In some embodiments, the single cell suspension is contacted with one or more (e.g. at least 1, 2, 3) of FLT3 ligand, SCF, or IL-7, or any combination thereof. In some embodiments, one or more (e.g. at least 1, 2, 3) of FLT3 ligand, SCF, or IL-7, or any combination thereof, is provided at a concentration that is, is about, is at least, is at least about, is not more than, or is not more than about, 0, 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, or 200 ng/mL, or any concentration within a range defined by any two of the aforementioned concentrations, for example, 0 to 200 ng/mL, 5 to 10 ng/ml, 20 to 100 ng/ml, or 100 to 200 ng/mL. In some embodiments, the liver organoid is contacted with one or more (e.g. at least 1, 2, 3) of FLT3 ligand, SCF, or IL-7, or any combination thereof, for a number of days that is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 14 days, 1 to 15 days, or 5 to 20 days. In some embodiments, isolating the lymphoid cells is performed by sorting for a cell population from the single cell suspension. In some embodiments, the lymphoid cells are isolated from the single cell suspension after culturing for a number of days that is, is about, is at least, is at least about, is not more than, or is not more than about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days within a range defined by any two of the aforementioned number of days, for example, 10 to 50 days, 15 to 40 days, 20 to 30 days, 10 to 30 days, or 20 to 50 days. In some embodiments, the B1 cells are CD20⁺CD43⁺CD27⁺CD70⁻. In some embodiments, isolating the B1 cells is performed by sorting for a cell population from the single cell suspension that is one or more (e.g. at least 1, 2, 3) of CD20, CD43, or CD27, or any combination thereof. In some embodiments, the B1 cells have autoimmune properties or innately express surface IgM, or both. In some embodiments, the B1 cells are not found in UCB cells. In some embodiments, the B1 cells are isolated from the single cell suspension after culturing for a number of days that is, is about, is at least, is at least about, is not more than, or is not more than about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days within a range defined by any two of the aforementioned number of days, for example, 10 to 50 days, 15 to 40 days, 20 to 30 days, 10 to 30 days, or 20 to 50 days.

In some embodiments, methods of expanding a population of hematopoietic stem cells are provided. In some embodiments, the hematopoietic stem cells in the fetal liver, or liver organoid, or both, are associated with Nestin portal vessels. In some embodiments, the liver organoid expresses stem cell factor (SCF) or thrombopoietin (TPO), or both, which induces HSC expansion and maintenance. In some embodiments, the method comprises culturing the population of hematopoietic stem cells in contact with a liver organoid. In some embodiments, the liver organoid comprises a hematopoietic niche environment. In some embodiments, the liver organoid is the liver organoid produced or isolated by any one of the methods described herein. In some embodiments, culturing the population of hematopoietic stem cells comprises contacting the population of hematopoietic stem cells with one or more cytokines. In some embodiments, culturing the population of hematopoietic stem cells comprises contacting the population of hematopoietic stem cells with SCF or TPO, or both. In some embodiments, SCF or TPO, or both, is provided at a concentration that is, is about, is at least, is at least about, is not more than, or is not more than about, 0, 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, or 200 ng/ml, or any concentration within a range defined by any two of the aforementioned concentrations, for example, 0 to 200 ng/mL, 5 to 10 ng/ml, 20 to 100 ng/ml, or 100 to 200 ng/ml. In some embodiments, the population of hematopoietic stem cells is contacted with SCF or TPO, or both for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, or 25 days, or any number of days within a range defined by any two of the aforementioned number of days, for example, 1 to 25 days, 5 to 20 days, 10 to 20 days, 1 to 20 days, or 10 to 25 days. In some embodiments, the expanded population of hematopoietic stem cells maintains pluripotency. In some embodiments, the expanded population of hematopoietic stem cells maintains pluripotency for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days within a range defined by any two of the aforementioned number of days, for example, 1 to 50 days, 5 to 40 days, 10 to 30 days, 1 to 30 days, or 20 to 50 days. In some embodiments, the population of hematopoietic stem cells is obtained from umbilical cord blood, mobilized peripheral blood, bone marrow, pluripotent stem cells, embryonic stem cells, fetal liver, or fetal spleen, or any combination thereof. In some embodiments, liver organoids that are differentiated with one or more (e.g. at least 1, 2, 3, 4) of RA, dexamethasone, Oncostatin M, or hepatocyte growth factor, or any combination thereof, do not support hematopoietic cells as well as undifferentiated, fetal-like liver organoids.

In some embodiments, the population of hematopoietic stem cells that are expanded are grafted into an individual. In some embodiments, the individual is a mouse. In some embodiments, the individual is an immunocompromised mouse. In some embodiments, the individual is a human. In some embodiments, the individual is an immunocompromised human. In some embodiments, the individual is an individual in need of hematopoietic stem cells. In some embodiments, the population of hematopoietic stem cells is injected into the individual. In some embodiments, the population of hematopoietic stem cells is injected intrafemorally. In some embodiments, the population of hematopoietic stem cells is injected with liver organoid cells. In some embodiments, the population of hematopoietic stem cells is injected after isolating the population of hematopoietic stem cells, where there are no liver organoid cells left. In some embodiments, the individual is also administered SCF or TPO, or both. In some embodiments, the population of hematopoietic stem cells engraft into the individual. In some embodiments, the individual in need of hematopoietic stem cells is in need for the treatment, inhibition, or amelioration of conditions including but not limited to aplastic anemia, Fanconi anemia, Diamond-Blackfan syndrome, sickle cell disease, thalassemia, paroxysmal nocturnal hemoglobinuria, Chediak-Higashi syndrome, chronic granulomatous disease, Glanzmann thrombasthenia, osteopetrosis, Gaucher disease, Niemann-Pick disease, mucopolysaccharidosis, glycoproteinoses, immune deficiencies, AIDS, ataxia telangiectasia, DiGeorge syndrome, severe combined immunodeficiency, Wiscott-Aldrich syndrome, Kostmann syndrome, Shwachman-Diamond syndrome, leukemia, acute myelogenous leukemia, acute lymphoblastic leukemia, hairy cell leukemia, chronic lymphocytic leukemia, myelodysplasia, Hodgkin disease, Non-Hodgkin disease, multiple myeloma, myeloproliferative neoplasm, myelofibrosis, polycythemia vera, chronic myelogenous leukemia, neuroblastoma, desmoplastic small round cell tumor, Ewing sarcoma, or choriocarcinoma, or any combination thereof.

In some embodiments, the population of hematopoietic stem cells is autologous or allogeneic to the liver organoid. In some embodiments, the population of hematopoietic stem cells is obtained from an individual. In some embodiments, the liver organoid is differentiated from cells obtained from an individual. In some embodiments, the liver organoid is produced from induced pluripotent stem cells derived from an individual. In some embodiments, the population of hematopoietic stem cells and liver organoid are obtained or derived from the same individual. In some embodiments, the individual is an individual in need of hematopoietic stem cells.

Described herein are methods of administering to an individual in need a population of hematopoietic stem cells. In some embodiments, the population of hematopoietic stem cells is an isolated population of hematopoietic stem cells. In some embodiments, the population of hematopoietic stem cells is expanded, produced, or isolated, or any combination thereof, according to any one of the methods described herein. In some embodiments, the population of hematopoietic stem cells is autologous or allogeneic to the individual in need. In some embodiments, the population of hematopoietic stem cells is autologous to the individual in need. In some embodiments, the population of hematopoietic stem cells is allogeneic to the individual in need.

Described herein are methods of administering to an individual in need a hemogenic endothelium population. In some embodiments, the hemogenic endothelium population is an isolated hemogenic endothelium population. In some embodiments, the hemogenic endothelium population is produced or isolated, or both, according to any one of the methods described herein. In some embodiments, the hemogenic endothelium population is autologous to the individual in need. In some embodiments, the hemogenic endothelium population is allogeneic to the individual in need.

Described herein are methods of administering to an individual in need lymphoid cells. In some embodiments, the lymphoid cells are isolated lymphoid cells. In some embodiments, the lymphoid cells are produced or isolated, or both, according to any one of the methods described herein. In some embodiments, the lymphoid cells are autologous to the individual in need. In some embodiments, the lymphoid cells are allogeneic to the individual in need. In some embodiments, the lymphoid cells comprise one or more of B cells, T cells, NK cells, or common lymphoid progenitor cells, or any combination thereof. In some embodiments, the B cells are B1 cells or B2 cells, or both.

Described herein are methods of administering to an individual in need B1 cells. In some embodiments, the B1 cells are isolated B1 cells. In some embodiments, the B1 cells are produced or isolated, or both, according to any one of the methods described herein. In some embodiments, the B1 cells are autologous to the individual in need. In some embodiments, the B1 cells are allogeneic to the individual in need.

Described herein are embodiments of a cell composition. In some embodiments, the cell composition comprises the liver organoid produced by any one of the methods described herein. In some embodiments, the cell composition comprises the hemogenic endothelium population produced or isolated by any one of the methods described herein. In some embodiments, the cell composition comprises the hematopoietic stem cells produced or isolated by any one of the methods described herein. In some embodiments, the cell composition comprises the B1 cells produced or isolated by any one of the methods described herein. In some embodiments, the cell composition comprises any combination of the other cell compositions described herein. In some embodiments, the cell composition comprises one or more (e.g. at least 1, 2, 3, 4) of the liver organoid, hemogenic endothelium population, hematopoietic stem cells, or B1 cells, or any combination thereof disclosed herein. In some embodiments, the cell composition comprises a population of hematopoietic stem cells and a liver organoid. In some embodiments, the population of hematopoietic stem cells is prepared or isolated, or both, according to any one of the methods described herein. In some embodiments, the liver organoid is prepared or isolated, or both, according to any one of the methods described herein. In some embodiments, the population of hematopoietic stem cells and liver organoid are from, obtained from, or derived from the same individual. In some embodiments, the population of hematopoietic stem cells and liver organoid are from, obtained from, or derived from different individuals.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1. Material and Methods

hPSC Maintenance

Human iPSC lines were maintained as described previously (Takebe et al., 2015; Takebe et al. 2014). Undifferentiated hiPSCs were maintained on feeder-free conditions in StemFit medium (StemCell Technologies) on plates coated with Laminin (1% iMatrix-511 silk in PBS) for 2 hours at 37° C. iPSCs were seeded at 8×10⁴ cells/well of 6-well cell culture treated plates (BD Falcon) in StemFit media supplemented with 100 ng/ml FGF2 and 10 nM ROCK inhibitor Y-27632 at 37° C. in 5% CO₂.

### Definitive Endoderm Induction

Differentiation of human iPSCs into definitive endoderm was induced using previously described methods with slight modifications (Spence et al. 2011). Briefly, colonies of human iPSCs were isolated in Accutase (Thermo Fisher) in Dulbecco's phosphate buffered saline (DPBS, Life Technologies) and 150,000 cells/mL were plated on Matrigel of a 1/30 dilution on tissue culture plates in RPMI 1640 medium (Life Technologies) supplemented with 100 ng/mL Activin A (R&D Systems) and 50 ng/ml bone morphogenic protein 4 (BMP4, R&D Systems) at day 1, and 100 ng/ml Activin A and 2% fetal calf serum (FCS) at day 3. On days 4-6, cells were cultured in Advanced DMEM (Thermo Fisher) with B27 (Life Technologies) and N2 (Gibco) supplements and additionally supplemented with 500 ng/mL fibroblast growth factor 4 (FGF4, R&D Systems) and 3 μM CHIR99021 (Stemgent). Cells were maintained at 37° C. in 5% CO₂, and the medium was replaced every day. Spheroids appeared on the plate at day 7 of differentiation.

### Human Liver Organoid (HLO) Formation

Day 7 spheroids were collected by manual pipetting and pelleted by centrifugation. Media was aspirated and cells were embedded in 100% Matrigel by gentle pipetting to homogenize the cell pellet. Optionally, a Notch activator or ligand is added to the Matrigel (e.g. 10 μg/mL of DLL4). Alternatively, optionally, UCB cells are added to the Matrigel for co-culture with organoids (e.g. 1,000 UCB cells per 75 μL of Matrigel). 250 μL Matrigel (Corning) was used per well of a 24-well plate of definitive endoderm culture. 75 μL Matrigel drops were made, 1 drop per each well of a fresh 24-well plate (VWR). The plates were placed at 37° C. in an atmosphere of 5% CO₂/95% air for 5-15 minutes. After the Matrigel was solidified, Advanced DMEM/F12 with 1×B27, 1×N2, 2 mM L-glutamine, 15 mM HEPES, and 1× penicillin/streptomycin was added to the wells. This media was supplemented with 20 ng/mL BMP4 and 10 ng/mL FGF2 for 4 days, and then the BMP4 and FGF2 were removed.

### Albumin ELISA

To measure albumin secretion level of HLOs, 200 μL of culture supernatant was collected from HLOs embedded in Matrigel. The culture supernatants were collected 24 hours after media changes and stored at −80° C. until use. The supernatant was assayed with a Human Albumin ELISA Quantitation Set kit (Bethyl Laboratories, E80-129) according to manufacturer's instructions.

### Histology

Samples in Matrigel drops were collected, fixed in 4% paraformaldehyde and embedded in paraffin. Sections were subjected to hematoxylin and eosin (H&E) and immunohistochemical staining. The following primary antibodies were used: anti-human albumin (Sigma Aldrich, A668), anti-human CD34 (Roche, 760-2620), anti-human CD45 (Roche, 760-2505).

### Flow Cytometry

Organoids were isolated from Matrigel drops and washed with 1×PBS. HLOs were dissociated to single cells by treatment with Trypsin-EDTA (0.05%, with phenol red, Gibco) for 10 minutes. Cells were washed in DMEM/F12 with 10% FBS and suspended in PBS with 2% FBS. After filtration (e.g. through a 40-50 μm nylon filter), the single cells were subjected to flow cytometry with the following antibodies: anti-CD45-APC (Miltenyl, 130-098-143), anti-CD34-APC/Cy7 (BioLegend, 343513), anti-CD11b-PE/Cy7 (BioLegend, 301322), anti-CD19-BV421 (BioLegend, 302233), anti-CD27-AF700 (BioLegend, 356415), anti-CD43-FITC (BioLegend, 315204). For mouse engraftment experiments, anti-mouse CD45-BV421 (BioLegend, 103133) was used.

### Colony Forming Cell (CFC) Assay

150,000 cells/mL were plated into 3 mL of MethoCult SF H4436 serum-free methylcellulose-based medium (StemCell Technologies). The mixture was evenly distributed into 60 mm dishes and maintained in a humidified chamber at 37° C. for 14 days followed by examination by light microscopy. The media used contains insulin, transferrin, SCF, IL-3, IL-6, EPO, G-CSF, and GM-CSF as cytokines. SCF, IL-3, IL-6, G-CSF, and GM-CSF are supplemented at 100 ng/mL, EPO is supplemented at 1-8 IU/mL (approximately 10-100 ng/mL).

### Cytospin and Giemsa Stain

Cells from CFC assays were collected from methylcellulose media by washing with PBS and 7,000 cells were applied as a thin layer on glass slides using a Cytospin centrifuge (500 rpm for 10 minutes). Slides were air dried and stained with a Wright-Giemsa staining kit (Thermo Fisher) according to manufacturer's instructions, followed by examination by light microscopy.

### Mouse Transplantation

NSG-SGM3 (NSGS, NOD-scid IL2Rgnull-3/GM/SF) mice were bred and housed in an animal care facility. Animal experiments were performed in accordance with institutional guidelines approved by an animal care committee. Mice were sub-lethally immune ablated with Busulfan (30 μg/g of body weight in 10 μL volume) administered by IP injection 24 hours prior to cell injection. Cells were intrafemorally injected. Organoid cultures were co-cultured with umbilical cord blood (UCB) CD34+ cells and medium was supplemented with stem cell factor (SCF) and thrombopoietin (TPO) for 12 days. 2× 106 HLO+UCB cells or 2×10³ UCB CD34+ cells only were injected into mice. After 7 weeks, femoral bone marrow and blood were collected and analyzed by flow cytometry.

### RNA Isolation and RT-qPCR

RNA was isolated using the RNeasy mini kit (Qiagen). Reverse transcription was carried out using the High-Capacity cDNA Reverse Transcription Kit (Thermo Fisher) according to manufacturer's protocol. qPCR was carried out in duplicate using TaqMan Gene Expression Master Mix (Applied Biosystems) and probes from the Universal Probe Library (Roche) on a QuantStudio 3 Real-Time PCR System (Thermo Fisher). All primer and probe information for each target gene was obtained from the Universal Probe Library Assay Design Center (accessible on the world wide web at qpcr.probefinder.com/organism.jsp). The primers are as follows:

Alpha-fetoprotein (AFP) Forward:
(SEQ ID NO: 1)
tgtactgcagagataagtttagctgac
AFP Reverse:
(SEQ ID NO: 2)
tccttgtaagtggcttcttgaac
Albumin (ALB) Forward:
(SEQ ID NO: 3)
gtgaggttgctcatcggttt
ALB Reverse:
(SEQ ID NO: 4)
gagcaaaggcaatcaacacc
CD34 Forward:
(SEQ ID NO: 5)
ggcacagctggaggtcttat
CD34 Reverse:
(SEQ ID NO: 6)
tgttcaggcatcagagaagtg
Hemoglobin subunit gamma-1 (HBGI) Forward:
(SEQ ID NO: 7)
tggatcctgagaacttcaagc
HBG1 Reverse:
(SEQ ID NO: 8)
gccactgcagtcaccatct
HES1 Forward:
(SEQ ID NO: 9)
gaagcacctccggaacct
HES1 Reverse:
(SEQ ID NO: 10)
gtcacctcgttcatgcactc
HES5 Forward:
(SEQ ID NO: 11)
cccggggttctatgatatttg
HES5 Reverse:
(SEQ ID NO: 12)
gccctgaagaaagtcctctaca

Accession numbers are available as HUGO Gene Nomenclature Committee IDs: AFP (HGNC: 317), ALB (HGNC: 399), CD34 (HGNC: 1662), HBG1 (HGNC: 4831), HES1 (HGNC: 5192), HES5 (HGNC: 19764), EPO (HGNC: 3415), TPO (THPO, HGNC: 11795), SCF (KIT ligand, KITLG, HGNC: 6343).

### Single Cell RNA Sequencing

HLOs were isolated from Matrigel drops and washed with 1×PBS. HLOs were dissociated to single cells by treatment with Trypsin-EDTA (0.05%, with phenol red, Gibco) for 10 minutes. Cells were washed in DMEM/F12 with 10% FBS and suspended in PBS with 2% FBS. Cells then underwent single cell RNA-seq library prep and were sequenced with the 10× Genomics Chromium platform and 2868 cells were recovered for analysis. Sequenced reads were processed using the Cell Ranger gene expression pipelines mkfastq and count, starting with demultiplexing and conversion of barcode and read data to fastq files. Raw reads were aligned to the Hg19 genome and filtered, creating gene-barcode matrices. Analysis were performed in AltAnalyze, where gene and cell clusters were identified by unsupervised analysis to identify predominant sample groups via expression clustering. Following removal of outliers, clusters were restricted to having >5 cells, a minimum Pearson correlation of 0.5, and a between-cluster fold change >4. Cell cycle effects were removed. Cluster-based heatmaps and corresponding tSNE plots were generated using Rv3.3.3. Cell cluster identity was determined through lineage correlation and ontological analysis in ToppGene (available on the world wide web at toppgene.cchmc.org). The tSNE plot was established by Loupe Cell Browser v1.0.5.

### Example 2. Parallel Differentiation of Hepatic Endoderm and Hemogenic Endothelium

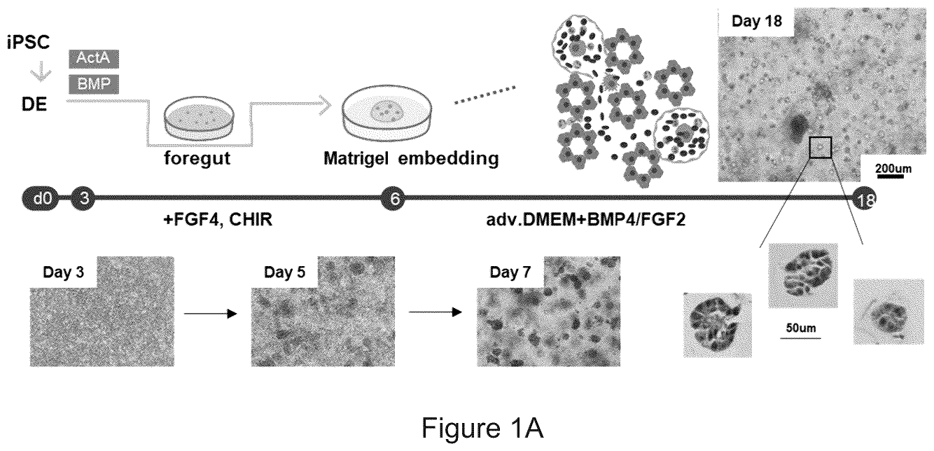

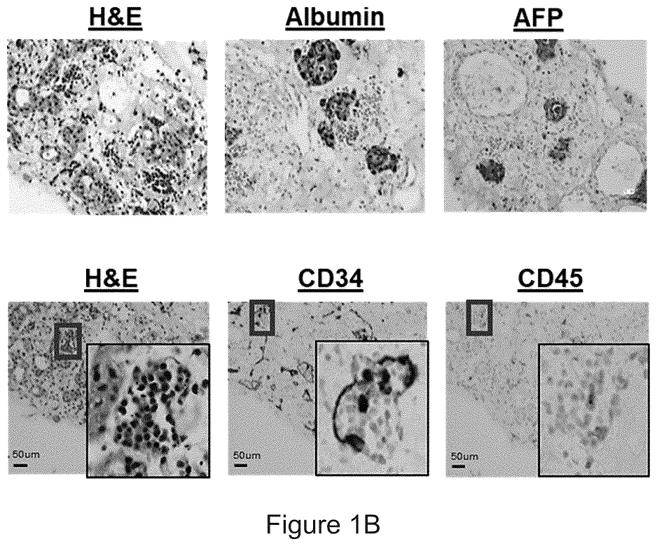

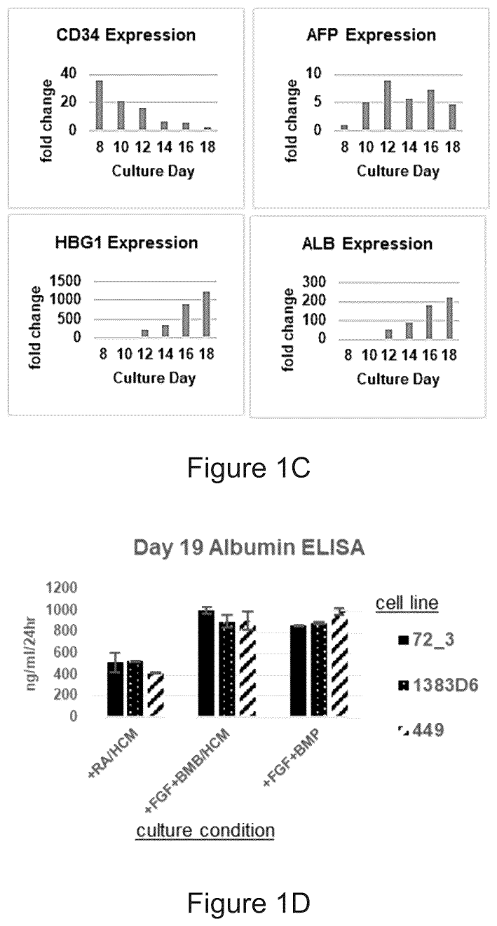

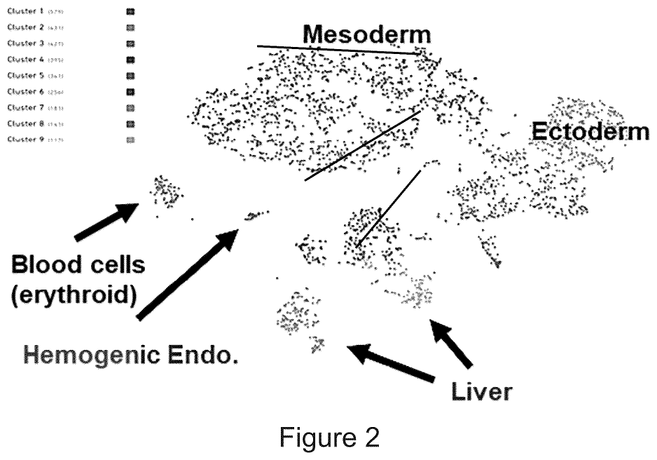

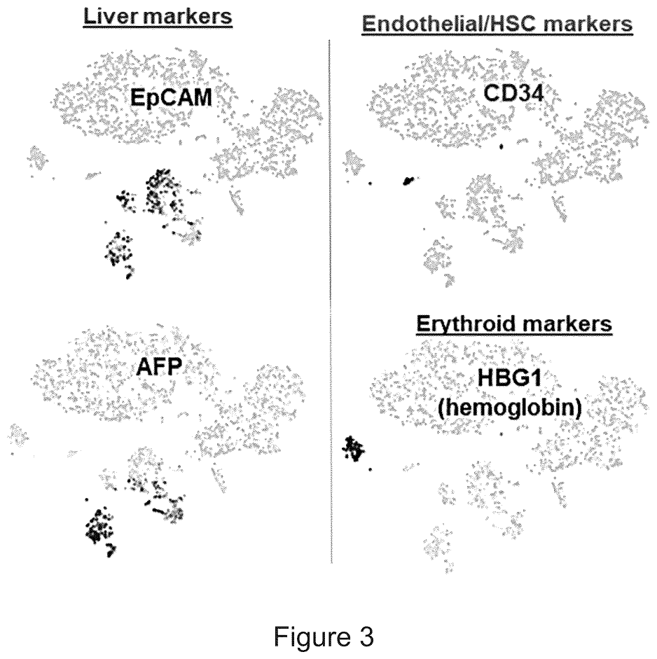

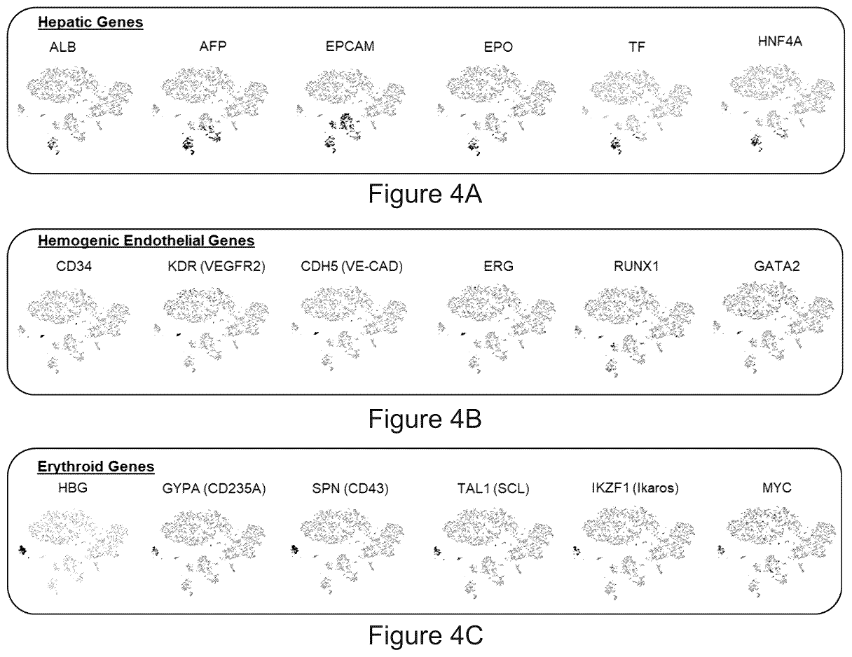

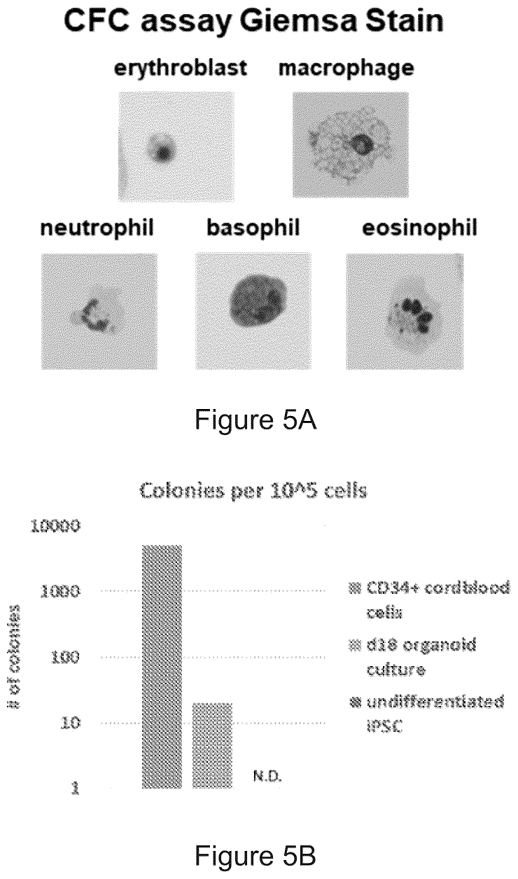

In order to mimic the fetal liver environment, iPSC-derived midgut spheroids were produced through endoderm induction in the presence of Activin A and embedded in Matrigel drops as described herein. Midgut spheroids were then cultured in the presence of BMP4 and FGF2, which are important in early hepatic specification. The resulting organoid cultures expressed hepatic genes, including albumin (ALB) and alpha-fetoprotein (AFP), and the endothelial marker CD34 (FIGS. 1A-D). The organoids also comprised a hematopoietic population that expressed fetal hemoglobin (as evidenced by the presence of hemoglobin gamma subunits). Additional analysis revealed that the organoid system contained a hemogenic endothelial population that was capable of producing erythrocytes without the addition of exogenous hematopoietic cytokines (FIG. 1A). This was further verified by single cell RNA sequencing (scRNA-seq) of day 14 organoid cultures. Expression profile analysis showed that at this time point, there were three populations of interest, in addition to a large mesenchyme population expressing mesoderm and ectoderm markers (FIGS. 2, 3). The clusters of interest comprised a hepatic population, an erythroid population, and a hemogenic endothelium population (FIGS. 4A-C). These hemogenic endothelial cells were further analyzed through colony forming cell (CFC) assays, in which cells from organoid cultures were dispersed into a single cell suspension and plated onto methylcellulose-based media with cytokines. The plated cells produced both erythroid and myeloid colonies, with a peak colony formation occurring around organoid culture day 14 (FIG. 5A-B).

### Example 3. Emergence of Notch Dependent Hemogenic Endothelium

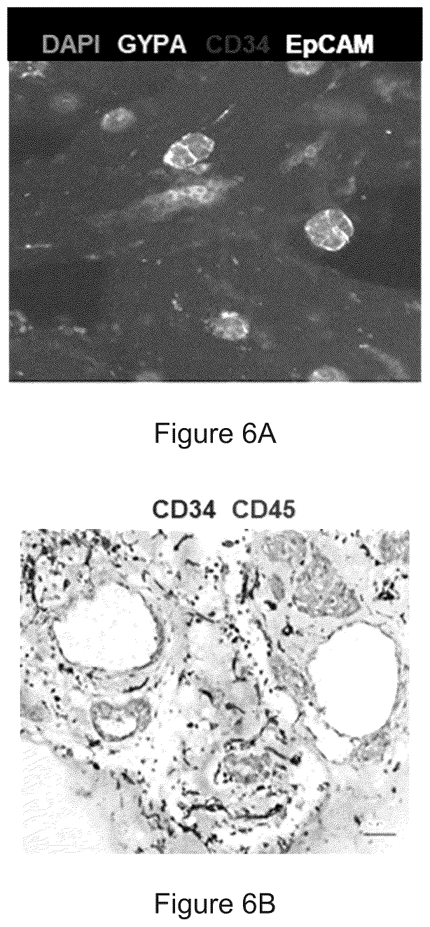

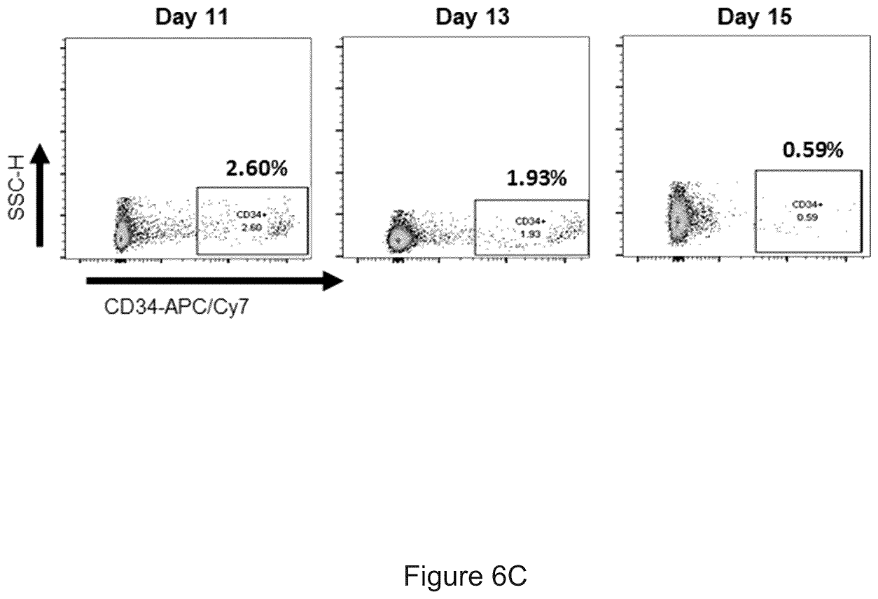

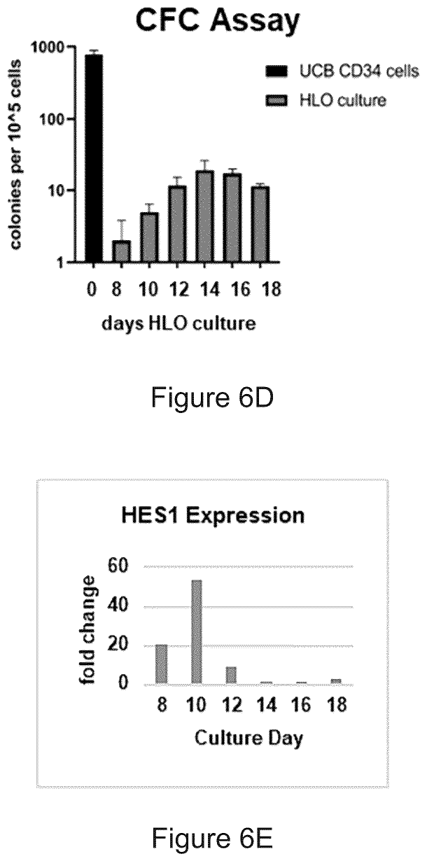

Histological examination of liver organoid cultures revealed epithelial cell adhesion molecule (EpCAM)-expressing cells alongside CD34⁺ endothelium with adjacent erythroid cells expressing glycophorin A (CD235a, GYPA) as well as CD45 expressing hematopoietic cells (FIG. 6A-B). Time course analysis of CD34-expressing cells revealed that the number of CD34⁺ cells decreased prior to a peak in CFC assay activity (FIG. 6C-D).

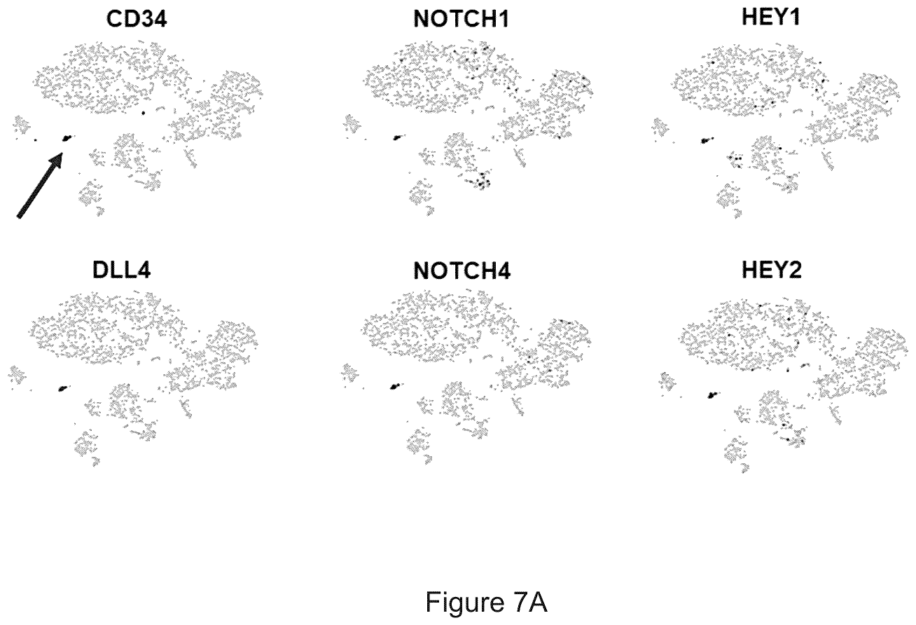

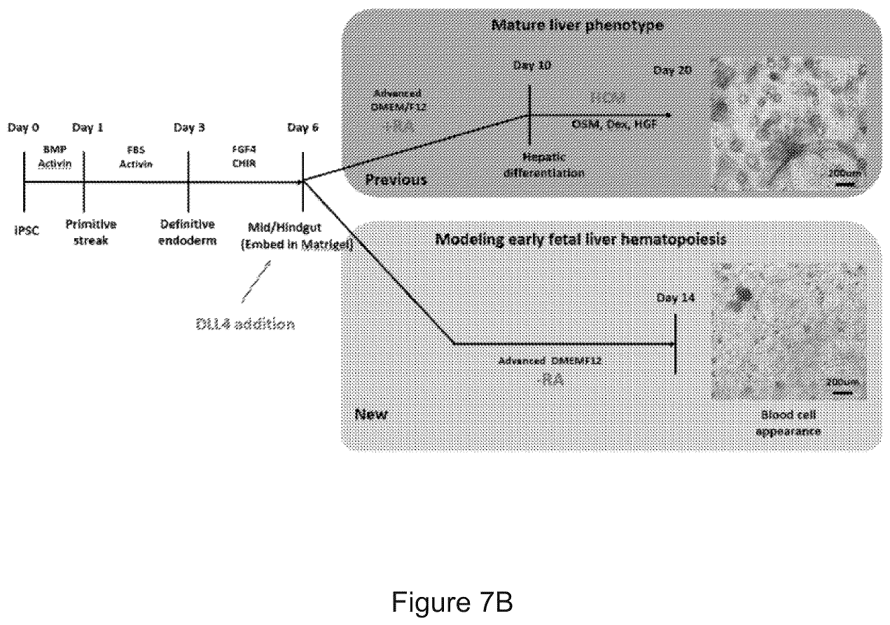

Notch signaling has previously been shown to be important in establishing definitive hematopoiesis in hemogenic endothelium. Specifically, Notch receptors Notch1 and Notch4, along with Notch ligands JAG1, JAG2, and DLL4 are expressed by aortic endothelial cells and are essential for early hematopoiesis through hemogenic endothelium. During this process, endothelial cells expressing the receptor Notch1 or Notch4 can interact with adjacent endothelial cells expressing the ligand DLL4 leading to increased expression of downstream target genes such as HES1/5, HEY1/2, and GATA2. A sharp increase in Notch signaling is evidenced by increased HES1 expression levels that peak around day 10 of organoid culture. This peak is immediately prior to the peak in hemogenic activity demonstrated by CFC assays, which peak around day 12 to 14 (FIGS. 6D-E). Flow cytometry of organoid cells to detect CD34 showed a steady decrease in CD34 cell numbers as organoids differentiated (FIG. 6C). To better understand the population dynamics of the organoid systems described herein, day 14 organoid cultures were analyzed by scRNA-seq. Closer analysis of the CD34 endothelial population revealed very specific expression of Notch signaling genes Notch1, Notch4, HEY1, HEY2, DLL4, and GATA2 (FIG. 7A, 4C).

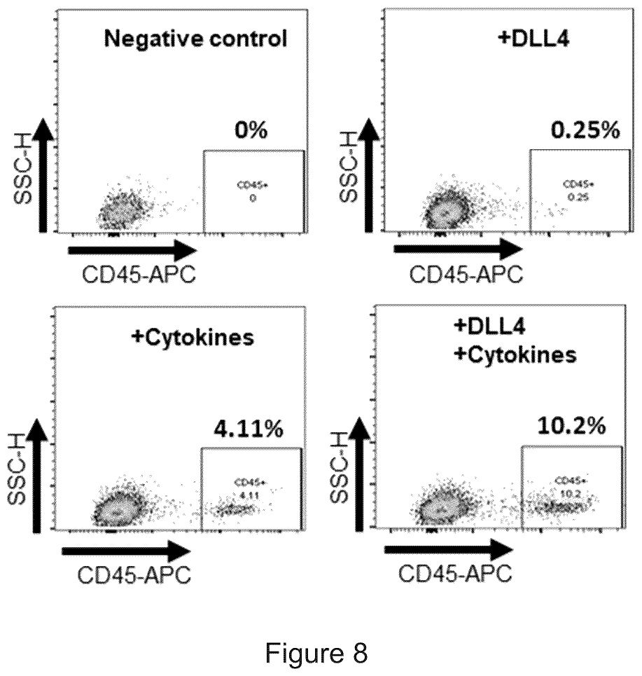

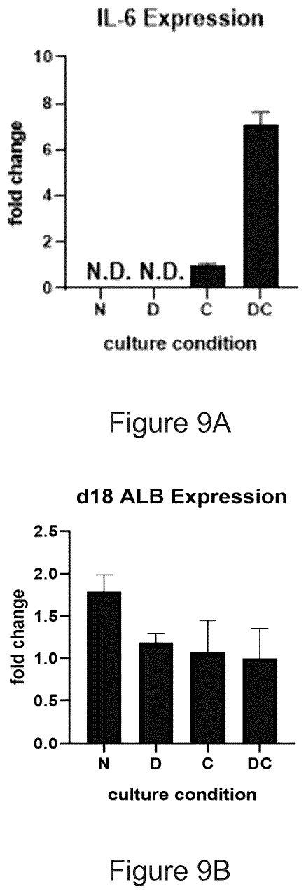

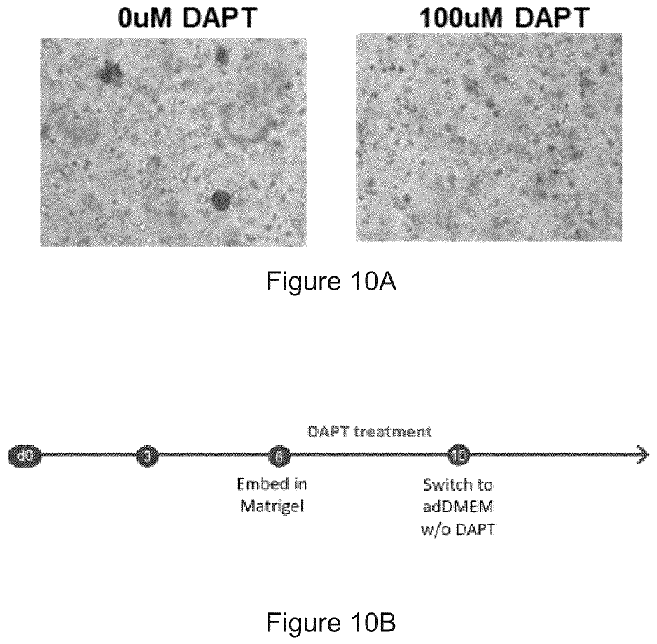

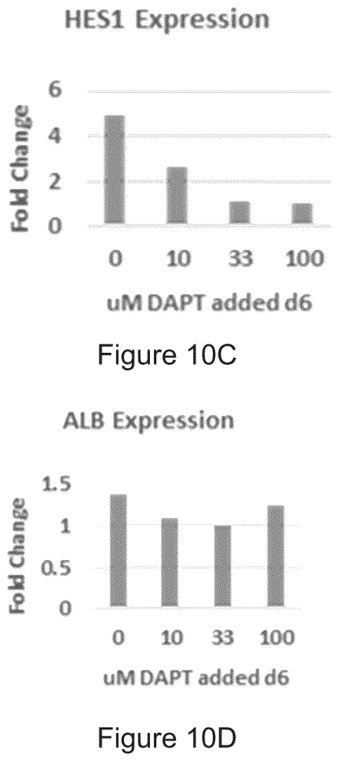

To test Notch dependency in the organoid culture system, the Notch ligand DLL4 (1-10 μg/mL) was added to the Matrigel drops during organoid seeding, and the organoid growth medium was supplemented with hematopoietic cytokines IL-3, IL-34, and GM-CSF (100 ng/ml each) (FIG. 7B). Addition of DLL4 increased the efficiency of cytokines in producing CD45⁺ hematopoietic cells (FIG. 8). This resulted in a significant increase in IL-6 expression with only a modest decrease in albumin expression (FIG. 9A-B). Conversely, organoid cultures treated with the γ-secretase inhibitor DAPT to inhibit Notch activity resulted in a decrease in visible erythrocytes and HES1 expression while maintaining albumin expression (FIG. 10A-D).

### Example 4. Creation of B1 Cells from Organoid Cultures

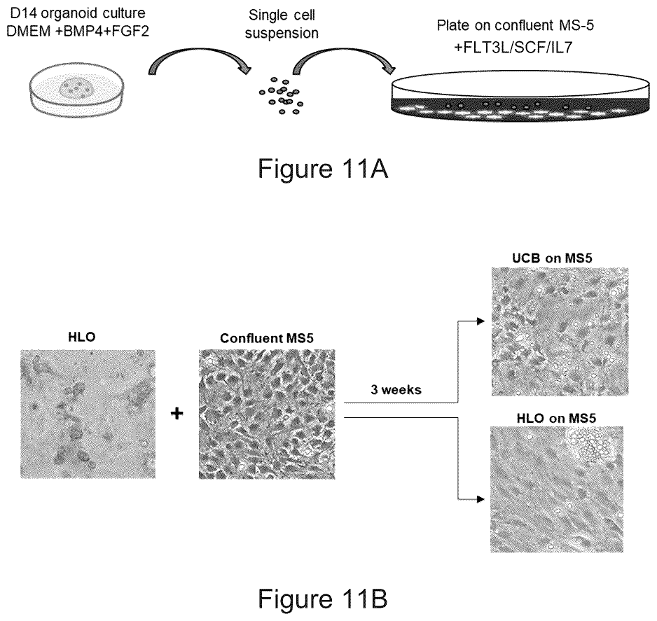

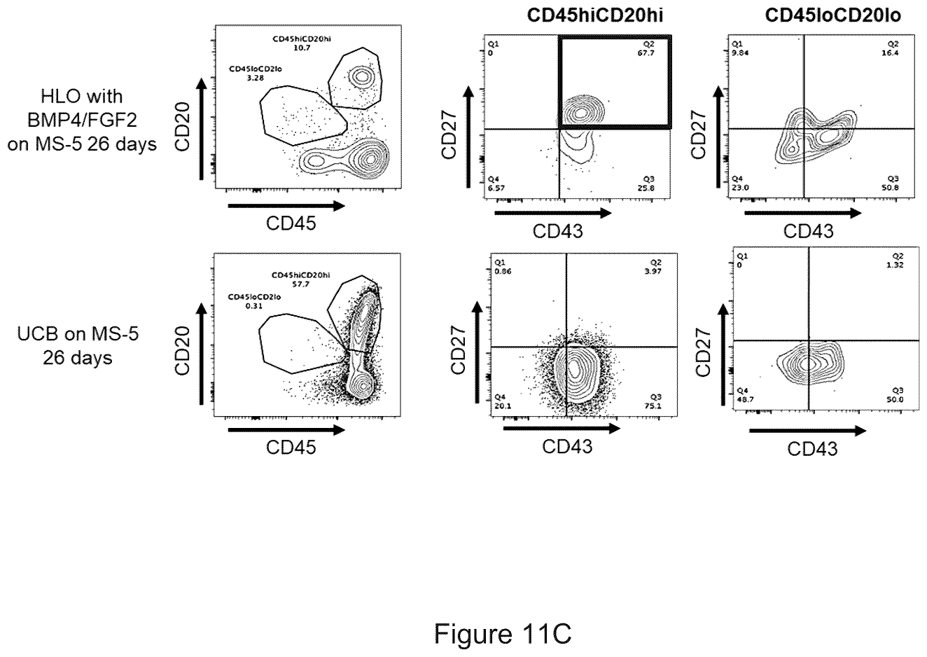

Definitive hematopoiesis is characterized by the production of both myeloid and lymphoid lineages. In order to examine the lymphoid potential of the organoid cultures described herein, cells were collected around organoid culture day 14 and plated onto confluent MS-5 murine stromal cells to differentiate into B cells (FIG. 11A-B). MS-5 cells are cultured in media supplemented with 5 ng/mL FLT3 ligand (FLT3L), 100 ng/ml SCF, and 5 ng/ml IL-7. During development, the fetal liver is a source of the unique B1 cell population. These cells are different from adult (B2) cells in that they arise independently from HSCs. B1 cells have autoimmune properties, innately express surface IgM, and have been described to be CD20⁺CD43⁺CD27⁺CD70⁻. Cells from organoids were compared to UCB cells in their production of B cells from MS-5 co-cultures. Interestingly, while both organoids and UCB were able to exhibit B cell differentiation, organoid-derived B cell populations included a unique B1 cell phenotype that was not able to be recreated from UCB cells. FACS analysis revealed a population of CD20⁺CD43⁺CD27⁺ cells (FIG. 11C).

### Example 5. Creation of a Hematopoietic Niche Environment

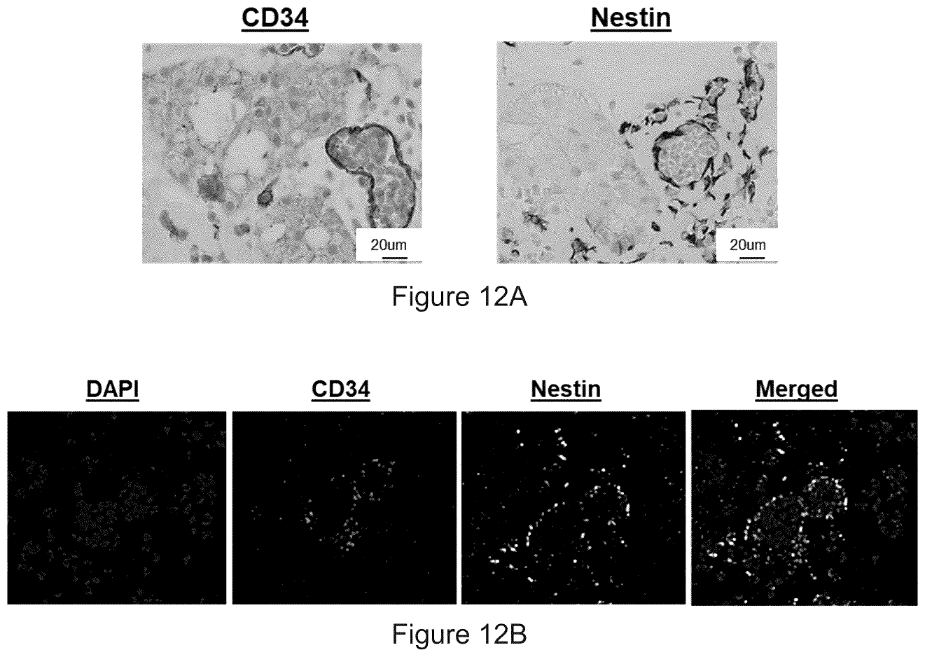

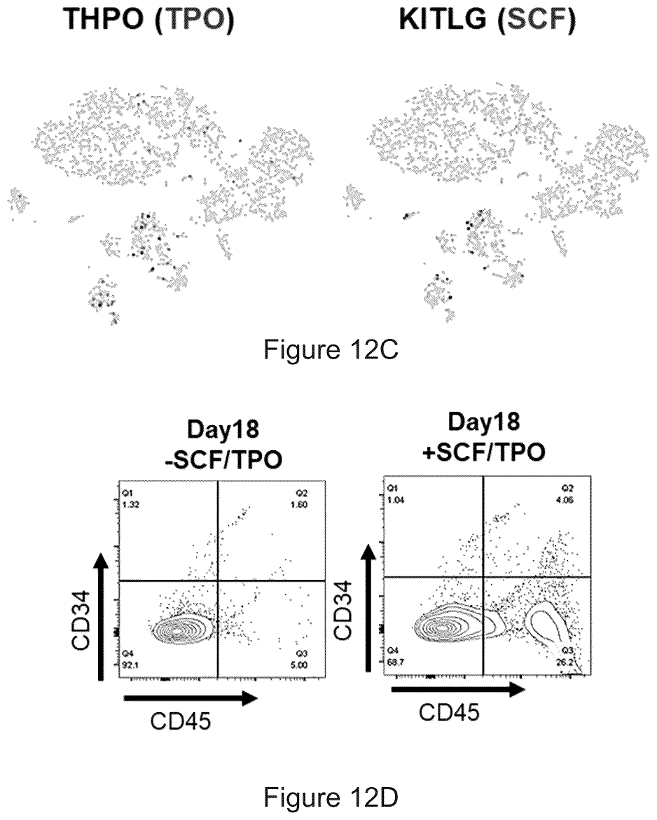

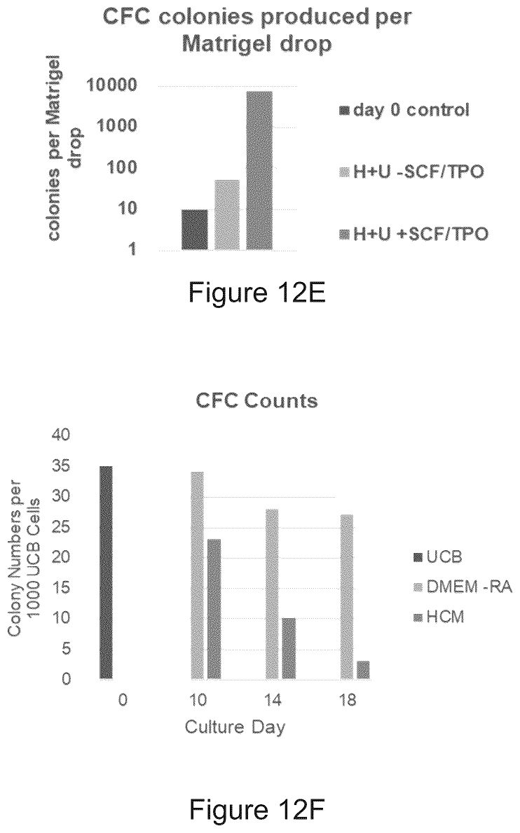

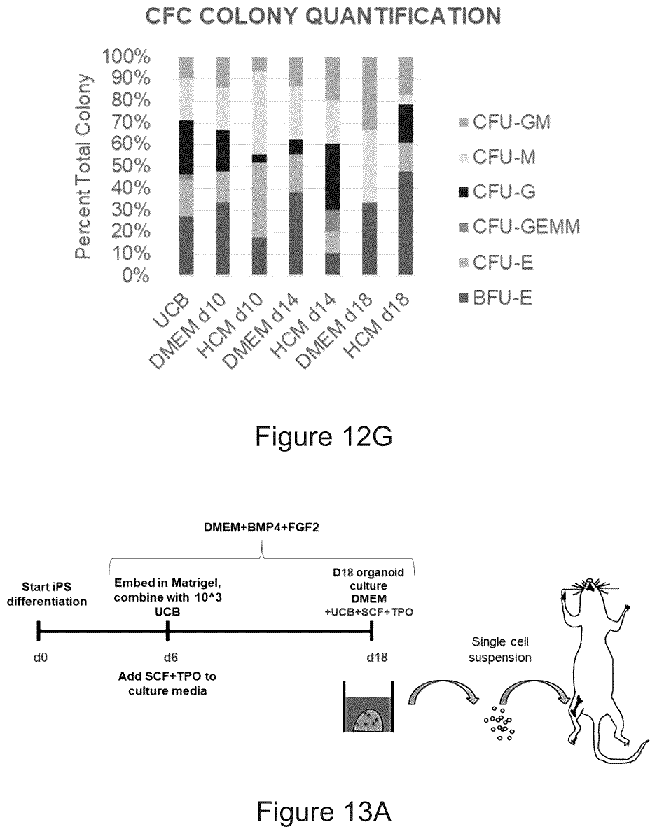

Hematopoietic stem and progenitor cells exist within a specific and complex niche environment that is not yet fully understood, particularly during fetal development during which HSCs in the fetal liver undergo massive expansion. In order to better understand the origin of the hematopoietic cells in the organoid cultures described herein, organoid cells were analyzed for hematopoietic niche markers. HSCs in the fetal liver are associated with Nestin⁺ portal vessels. Within the organoid cultures, pockets of CD34⁺ cells were also seen in close association with Nestin staining, and scRNA-seq also showed a population of CD34/Nestin co-expressing cells (FIGS. 12A-B, 4A-D). Interestingly, scRNA-seq also revealed that cells clustered with hepatic populations expressed stem cell factor (SCF) and thrombopoietin (TPO) (FIG. 12C), which are essential for HSC expansion and maintenance and are often used in attempts to expand HSCs ex vivo.

To further test the niche environment in organoid cultures, CD34 human UCB cells were added to the Matrigel drops during organoid seeding. Exogenous recombinant SCF and TPO was added to the growth medium to improve the efficiency of hematopoietic cell production and maintenance. Addition of 100 ng/mL SCF and 10 ng/ml TPO resulted in a significant increase in CD34⁺ and CD45⁺ cell production in the organoid cultures as well as an increase in the number of colonies formed (FIGS. 12D-E).

To determine if these results were due to the presence of a hematopoietic niche or merely a contaminating mesoderm population resulting in hematopoietic differentiation, the ability of UCB cells to form colonies after mixing with the organoid cultures was examined. Promotion of hepatic differentiation of the organoids with the addition of retinoic acid (RA) followed by hepatocyte culture medium (HCM, Lonza), optionally supplemented with 0.1 μM dexamethasone, 10 ng/ml Oncostatin M, and 10 ng/mL hepatocyte growth factor (HGF), resulted in a steady decline of hematopoietic colony forming ability. Conversely, the protocols described herein modified to limit hepatic differentiation maintains colony forming cell presence as well as their diverse differentiation potential, demonstrating the existence of a unique niche that models the transient stage of fetal liver hematopoiesis and relies on the presence of an immature hepatic population (FIGS. 12F-G).

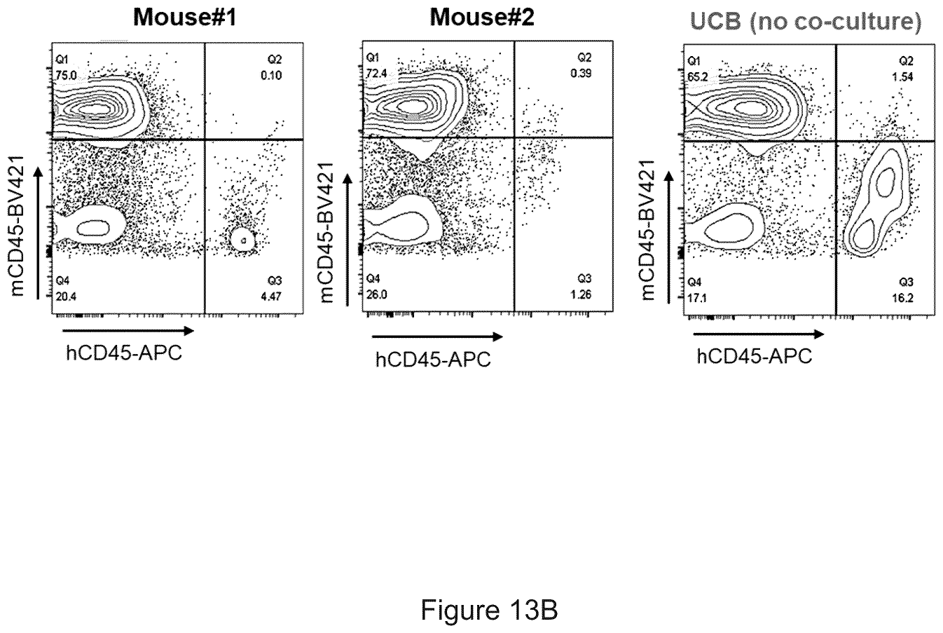

UCB cells are capable of engrafting into immunocompromised recipients and re-establishing the complete hematopoietic system. However, a limiting factor in the potential therapeutic application of these cells is their inability to be cultured or expanded in vitro. In order to further analyze the niche environment of the organoid systems described herein, the engraftment of UCB cells after co-culture with organoids was tested. UCB cells were combined with organoids in Matrigel drops and the growth medium was supplemented with SCF and TPO at organoid culture day 6. After 12 days, UCB and organoid cells were collected into a single cell suspension and intrafemorally injected into the bone marrow of NSGS mice. Seven weeks later, mice showed engraftment of human specific CD45 cells (FIGS. 13A-B). The resulting engraftment was around 3% engraftment after co-culturing the UCB cells with organoids compared to around 16% engraftment without co-culture. Although this engraftment is decreased compared to fresh cord blood cells, UCB cultured in the same conditions without organoids immediately lost their engraftment potential. Thus, this provides a new system for culturing HSC while maintaining engraftment capability.

Shown herein, a novel fetal liver organoid system derived from human iPSCs is capable of giving rise to both hepatic and hematopoietic cells in a symbiotic co-differentiation system. Hematopoietic cells in this system arise from a hemogenic endothelium population and differentiate into both myeloid and lymphoid lineages in the presence of cytokines. Co-culture of organoid cells with MS-5 mouse stromal cells give rise to a fetal B1-like cell population as identified by flow cytometry. Although there was only limited engraftment after co-culture of UCB cells with the organoids, this system provides evidence for a novel way to maintain HSC in culture through mimicking their natural environment.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Sugimura R, Jha D K, Han A, et al. Haematopoietic stem and progenitor cells from human pluripotent stem cells. Nature. 2017; 545 (7655): 432-438. doi: 10.1038/nature22370
Lis R, Karrasch C C, Poulos M G, et al. Conversion of adult endothelium to immunocompetent haematopoietic stem cells. Nature. 2017; 545 (7655): 439-445. doi: 10.1038/nature22326
Wilkinson A C, Ishida R, Kikuchi M, et al. Long-term ex vivo haematopoietic-stem-cell expansion allows nonconditioned transplantation. Nature. 2019; 571 (7763): 117-121. doi: 10.1038/s41586-019-1244-x
Rothstein T L, Griffin D O, Holodick N E, Quach T D, Kaku H. Human B-1 cells take the stage. Ann N Y Acad Sci. 2013; 1285:97-114. doi: 10.1111/nyas.12137
Montecino-Rodriguez E, Leathers H, Dorshkind K. Identification of a B-1 B cell-specified progenitor. Nat Immunol. 2006; 7 (3): 293-301. doi: 10.1038/ni1301
McCracken K W, Aihara E, Martin B, et al. Wnt/β-catenin promotes gastric fundus specification in mice and humans. Nature. 2017; 541 (7636): 182-187. doi: 10.1038/nature21021
Spence J R, Mayhew C N, Rankin S A, et al. Directed differentiation of human pluripotent stem cells into intestinal tissue in vitro. Nature. 2011; 470 (7332): 105-109. doi: 10.1038/nature09691
Si-Tayeb K, Noto F K, Nagaoka M, et al. Highly efficient generation of human hepatocyte-like cells from induced pluripotent stem cells. Hepatology. 2010; 51 (1): 297-305. doi: 10.1002/hep.23354
Uenishi G I, Jung H S, Kumar A, et al. NOTCH signaling specifies arterial-type definitive hemogenic endothelium from human pluripotent stem cells. Nat Commun. 2018; 9. doi: 10.1038/s41467-018-04134-7
Robert-Moreno À, Espinosa L, Pompa J L de la, Bigas A. RBPjκ-dependent Notch function regulates Gata2 and is essential for the formation of intra-embryonic hematopoietic cells. Development. 2005; 132 (5): 1117-1126. doi: 10.1242/dev.01660
Robert-Moreno À, Guiu J, Ruiz-Herguido C, et al. Impaired embryonic haematopoiesis yet normal arterial development in the absence of the Notch ligand Jagged1. EMBO J. 2008; 27 (13): 1886-1895. doi: 10.1038/emboj.2008.113
Griffin D O, Holodick N E, Rothstein T L. Human B1 cells in umbilical cord and adult peripheral blood express the novel phenotype CD20+CD27+CD43+CD70−. J Exp Med. 2011; 208 (1): 67-80. doi: 10.1084/jem.20101499
Khan J A, Mendelson A, Kunisaki Y, et al. Fetal liver hematopoietic stem cell niches associate with portal vessels. Science. 2016; 351 (6269): 176-180. doi: 10.1126/science.aad0084
Ueda T, Tsuji K, Yoshino H, et al. Expansion of human NOD/SCID-repopulating cells by stem cell factor, Flk2/Flt3 ligand, thrombopoietin, IL-6, and soluble IL-6 receptor. J Clin Invest. 2000; 105 (7): 1013-1021.
Zhang C C, Kaba M, Iizuka S, Huynh H, Lodish H F. Angiopoietin-like 5 and IGFBP2 stimulate ex vivo expansion of human cord blood hematopoietic stem cells as assayed by NOD/SCID transplantation. Blood. 2008; 111 (7): 3415-3423. doi: 10.1182/blood-2007-11-122119

1.-45. (canceled)

46. An in vitro liver organoid that produces hematopoietic cells, wherein the organoid comprises a hematopoietic niche environment.

47. The in vitro liver organoid of claim 46, wherein the organoid is capable of producing erythrocytes without the addition of exogenous hematopoietic cytokines.

48. The in vitro liver organoid of claim 46, wherein the liver organoid comprises a CD34+ hemogenic endothelium population that differentiate into hematopoietic cells.

49. The in vitro liver organoid of claim 46, wherein the organoid comprises an immature hepatic population.

50. The in vitro liver organoid of claim 46, wherein the CD34+ hemogenic endothelium population originates from the mesenchyme of a foregut spheroid.

51. The in vitro liver organoid of claim 50, wherein foregut spheroid is obtained by culturing PSCs, iPSCs, or definitive endoderm.

52. The in vitro liver organoid of claim 46, wherein hematopoietic cells are hemogenic endothelium cells, hematopoietic stem cells, or hematopoietic progenitor cells, or any combination thereof.

53. The in vitro liver organoid of claim 46, wherein the hematopoietic cells are lymphoid cells.

54. The in vitro liver organoid of claim 53, wherein the lymphoid cells comprise one or more of B cells, T cells, NK cells, or common lymphoid progenitor cells.

55. The in vitro liver organoid of claim 46, wherein the hematopoietic cells do not exogenously express one or more of ERG, HOXA5, HOXA9, HOXA10, LCOR, RUNX1, SPI1, FOSB, and/or GFI1.

56. The in vitro liver organoid of claim 46, wherein the organoid expresses stem cell factor (SCF) or thrombopoietin (TPO), or both.

57. A hemogenic endothelium population isolated from the liver organoid of claim 46.

58. The hemogenic endothelium population of claim 57, wherein the hemogenic endothelium population is CD34+ or CD45+, or both.

59. The hemogenic endothelium population of claim 57, wherein the CD34+ hemogenic endothelium population originates from the mesenchyme of the foregut spheroid.

60. The hemogenic endothelium population of claim 57, wherein the foregut spheroid is obtained by culturing PSCs, iPSCs, or definitive endoderm.

61. A hematopoietic stem cell produced from the liver organoid of claim 46.

62. The hematopoietic stem cell of claim 62, wherein the hematopoietic stem cell maintains pluripotency for at least 10 days.

63. A cell composition comprising a population of hematopoietic stem cells (HSCs) and the liver organoid of claim 46.

64. A method for treating of a blood cancers, immune disorders, or gene defects in blood cells, the method comprising administering the organoid of claim 46 to an individual.

65. A method for maintaining hematopoietic stem cells comprising culturing hematopoietic stems cells with the organoid of claim 46.
